(19) United States
(12) Patent Application Publication
(10) Pub. No.: US 2026/0345758 A1
(43) Pub. Date: Nov. 5, 2026

(54) IMMUNOGENIC COMPOSITION

(71) Applicant: GRIFFITH UNIVERSITY, Nathan, Queensland (AU)

(72) Inventors: Michael GOOD, Nathan, Queensland (AU); Danielle STANISIC, Nathan, Queensland (AU)

(73) Assignee: GRIFFITH UNIVERSITY, Nathan, Queensland (AU)

(21) Appl. No.: 19/478,143

(22) PCT Filed: Apr. 24, 2024
(86) PCT No.: PCT/AU2024/050392

§ 371 Date: Oct. 23, 2025

(30) Foreign Application Priority Data
Apr. 24, 2023 (AU) 2023901207

(51) Int. Cl. A61K 39/015; A61K 39/00; A61P 37/04

(52) U.S. Cl. CPC A61K 39/015; A61P 37/04; A61K 2039/515; A61K 2039/54

(57) ABSTRACT

The present disclosure relates to the field of vaccines to protect against disease caused by erythrocytic organisms, such as Plasmodium sp., and methods of making and using same.

### CROSS-REFERENCE TO RELATED APPLICATIONS

The present application claims priority from Australian Provisional Patent Application No. 2023901207 filed on 24 Apr. 2023, the contents of which are incorporated herein by reference in their entirety.

### TECHNICAL FIELD

The present disclosure relates to the field of vaccines to protect against disease caused by erythrocytic organisms, such as Plasmodium sp., and methods of using same.

### BACKGROUND

An effective malaria vaccine will represent a cost-effective and sustainable addition to the currently available malaria control interventions. Malaria vaccines have attempted to target the different stages of malaria infection, typically referred to as the “sporozoite stage”, the “liver stage” and “blood-stage”. The liver stage occurs when sporozoites infect host hepatocytes, multiplying asexually and asymptomatically for a period of 8-30 days. Once in the liver, these organisms differentiate to yield thousands of merozoites which, following rupture of their host cells, escape into the blood and infect red blood cells, thus beginning the erythrocytic or “blood-stage” of the life cycle. Within the red blood cells, the parasites multiply further, again asexually, periodically breaking out of their hosts to invade fresh red blood cells. Several such amplification cycles occur. Thus, classical descriptions of waves of fever arise from simultaneous waves of merozoites escaping and infecting red blood cells.

Malaria vaccine development has focused primarily on the subunit approach in which a restricted number of parasite antigens are formulated with delivery systems and adjuvants to enhance immune responses against specific antigenic epitopes. However, this strategy has shown limited success with the vaccines being only moderately efficacious and immunity being short-lived (see, Genton et al., 2002; Sirima et al., 2011; Ewer et al., 2013; and Olotu et al., 2016).

These shortcomings have recently led to renewed interest in whole parasite (WP) vaccines and in particular WP blood-stage malaria vaccines. Genetically modified WP blood-stage malaria vaccines (see, Ting et al., 2008) and chemically attenuated WP blood-stage malaria vaccines (see, Good et al., 2013; and Raja et al., 2016) have been tested in murine models, and the latter in clinical studies (see, Stanisic et al., 2018), where they were shown to induce potent immune responses in malaria naive volunteers. An alternative approach is to use inactivated WP antigens, which have been adjuvanted (see, Su et al., 2003; and Pinzon-Charry et al., 2010).

One major drawback to WP vaccines (live or attenuated) is that the red cell membranes contained therein can lead to the induction of alloantibodies to red blood cell antigens following inoculation therewith. Patients having received such vaccines may then be susceptible to subsequent transfusion reactions and autoimmune haemolytic anaemia. This remains an issue even if the parasites are grown in blood type O, Rhesus negative (‘universal donor’) blood (see, Stanisic et al., 2018).

As such, there remains a clinical need for vaccines against erythrocytic organisms, such as malaria organisms, that induce significant protective immunity whilst also demonstrating an improved safety profile.

### SUMMARY

The present disclosure is based on the identification of a whole parasite vaccine composition that can function as a universal vaccine. One challenge in the development of whole erythrocytic parasite vaccines has been the inclusion of the host red blood cell (RBC) membrane in the composition. This is because anti-RBC antibodies can be induced as a result of the blood used to culture the erythrocytic parasite for the vaccine, which represents a clinically significant issue for both human and veterinary vaccines. Accordingly, the immunogenic composition described herein is suitably includes erythrocytes that have been treated with an antigen masking agent to thereby substantially mask RBC antigens thereon, which may advantageously avoid the production of anti-red blood cell antibodies and haemolytic sequelae in vaccinated subjects.

Accordingly, in a first aspect the present disclosure provides an immunogenic composition

In a first aspect, the present disclosure provides an immunogenic composition comprising:
a plurality of erythrocytes;
a plurality of erythrocytic organisms; and
an antigen masking agent conjugated, linked or otherwise associated with the erythrocytes.

Suitably, the antigen masking agent is or comprises a polyethylene glycol or a derivative thereof, such as methoxypolyethylene glycol (mPEG).

Suitably, the immunogenic composition further comprises an adjuvant. According to certain examples, the adjuvant is or comprises a lipid A glycolipid adjuvant or a derivative thereof, inclusive of a synthetic monophosphoryl 3-deacyl lipid A adjuvant or a native monophosphoryl lipid A adjuvant.

In some examples, the erythrocytes have been previously infected, such as infected in vitro or infected in vivo, with the erythrocytic organism.

Suitably, the erythrocytic organisms have been attenuated, inactivated and/or killed, such as chemically attenuated or inactivated. For some examples, the erythrocytic organisms have been chemically attenuated with a DNA binding agent, such as centanamycin, tafuramycin A or both.

In other examples, the erythrocytic organisms have been killed.

For particular examples, the erythrocytes are intact or substantially intact. In alternative examples, the erythrocytes are lysed or substantially lysed. According to other examples, the immunogenic composition includes a mixture of lysed erythrocytes and intact erythrocytes.

Suitably, the erythrocytic organisms are or comprise an intra-erythrocytic organism or an intra-erythrocytic parasite. In certain examples, the erythrocytic organism is selected from the group consisting of a Plasmodium sp., a Babesia sp., an Anaplasma sp., an Ehrlichia sp., a Trypanosoma sp., a Theileria sp., a Hepatozoon sp., a Mycoplasma sp., a Bartonella sp. and any combination thereof.

According to particular examples, the erythrocytic organisms are or comprise a Plasmodium sp. In such examples, the erythrocytic organisms are suitably selected from the group consisting of P. falciparum, P. malariae, P. vivax, P. ovale, P. knowlesi and any combination thereof.

Suitably, the erythrocytes are contained in or otherwise associated with a liposome.

In some examples, one or more antigens of the erythrocytes are substantially masked by the antigen masking agent.

In various examples, the immunogenic composition results in a reduced level of alloantibody production when administered to an antigen mismatched subject compared to a level of alloantibody production of a control immunogenic composition that does not comprise the antigen masking agent conjugated, linked or otherwise associated with the erythrocytes.

Suitably, the immunogenic composition is for use in a method of:
(a) eliciting an immune response in a subject; and/or
(b) preventing, treating or ameliorating an infection with an erythrocytic organism or a disease, disorder or condition associated therewith in a subject.

In a second aspect, the present disclosure provides a method of preventing, treating or ameliorating an infection with an erythrocytic organism or a disease, disorder or condition associated therewith in a subject, said method including the step of administering to the subject a therapeutically effective amount of the immunogenic composition of the first aspect.

In a third aspect, the present disclosure relates to a method of inducing an immune response in a subject, said method including the step of administering to the subject an effective amount of the immunogenic composition of the first aspect.

In a fourth aspect, the present disclosure provides for the use of the immunogenic composition of the first aspect in the manufacture of a medicament for:
(a) eliciting an immune response in a subject; and/or
(b) preventing, treating or ameliorating an infection with an erythrocytic organism or a disease, disorder or condition associated therewith in a subject.

In a fifth aspect, the present disclosure provides a method of producing an immunogenic composition, said method including the steps of:
(a) providing a plurality of erythrocytes that contain or are infected with an erythrocytic organism; and
(b) contacting the erythrocytes with an antigen masking agent for a period of time and under conditions to facilitate the antigen masking agent conjugating, linking or otherwise associating with the erythrocytes.

In particular examples, the present method further includes the step of inactivating, attenuating and/or killing the erythrocytic organisms.

In various examples, the present method further includes the step of adding an adjuvant to the immunogenic composition.

Suitably, the immunogenic composition is that of the first aspect.

In a sixth aspect, the present disclosure provides an immunogenic composition produced according to the method of the fifth aspect.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

FIG. 1: Treatment of mouse red blood cells with mPEG-cyanuric chloride reduces TER119 expression. Red blood cells (RBCs) derived from P. yoelii 17X infected BALB/c mice were treated with mPEG according to our standard protocol. Control RBCs were treated with phosphate buffered saline (PBS). Following treatment, RBCs were washed and stained with a fluorophore conjugated TER119 antibody. Using flow cytometry, TER119 expression on mPEG treated and PBS treated cells were compared.

FIG. 2: mPEG-cyanuric chloride treatment of P. yoelii 17X pRBCs in a whole blood-stage parasite malaria vaccine does not impact on vaccine efficacy but attenuates clinical scores. BALB/c mice were vaccinated sub-cutaneously (s.c.) with 3 doses of 10′ P. yoelii 17X parasitised red blood cells (pRBCs) or normal red blood cells (nRBCs) formulated with PHAD liposomes (1). Vaccine doses were administered 2 weeks apart. Prior to formulating the vaccine, P. yoelii 17X pRBCs and nRBCs were either treated with mPEG (treated) or PBS (untreated) and freeze thawed 6-8 times to lyse the RBC membranes. One month following administration of the final vaccine dose, mice were challenged intravenously (i.v.) with 10 live P. yoelii 17X pRBCs. Parasitemias (left graph) and clinical scores (right graph) were monitored post-challenge.

FIG. 3: mPEG-cyanuric chloride treatment of P. yoelii 17X pRBCs in a whole blood-stage parasite malaria vaccine (fresh or cryopreserved) does not impact on vaccine efficacy but attenuates clinical scores. BALB/c mice were vaccinated sub-cutaneously (s.c.) with 3 doses of 10⁷ P. yoelii 17X parasitised red blood cells (pRBCs) or normal red blood cells (nR.BCs) formulated with PHAD liposomes (1). Vaccine doses were either prepared fresh (top row of graphs) or flash frozen (bottom row of graphs) prior to administration and were administered 2 weeks apart. Prior to formulating the vaccine, P. yoelii 17X pRBCs and nRBCs were either treated with mPEG (treated) or PBS (untreated) and freeze thawed 6-8 times to lyse the RBC membranes. One month following administration of the final vaccine dose, mice were challenged intravenously (i.v.) with 10⁵ live P. yoelii 17X pRBCs. Parasitemias (left graphs), clinical scores (middle graphs) and hemoglobin levels (right graphs) were monitored post-challenge.

FIG. 4: mPEG-cyanuric chloride treatment of P. yoelii 17X pRBCs in a whole blood-stage parasite malaria vaccine does not impact on parasite-specific splenocyte proliferative responses. BALB/c mice were vaccinated sub-cutaneously (s.c.) with 3 doses of 10⁷ P. yoelii 17X parasitised red blood cells (pRBCs) or normal red blood cells (nRBCs) formulated with PHAD liposomes (1). Vaccine doses were administered 2 weeks apart. Prior to formulating the vaccine, P. yoelii 17X pRBCs and nRBCs were either treated with mPEG (treated) or PBS (untreated) and freeze thawed 6-8 times to lyse the RBC membranes. One month following administration of the final vaccine dose, spleens were removed, processed and splenocytes (4×10⁵/well) were incubated with P. yoelii 17X pRBCs (5×10⁵/well), nRBCs (5×10⁵/well), media or ConA for 72 hours at 37 °C/5% CO2. Tritiated thymidine was added for the last 18 hours of this incubation to enable assessment of splenocyte proliferation by radioisotope incorporation (1).

FIG. 5: mPEG-cyanuric chloride treatment of P. yoelii 17X pRBCs in a whole blood-stage parasite malaria vaccine does not significantly impact on parasite-specific cytokine responses. BALB/c mice were vaccinated sub-cutaneously (s.c.) with 3 doses of 10⁷ P. yoelii 17X parasitised red blood cells (pRBCs) or normal red blood cells (nRBCs) formulated with PHAD liposomes (1). Vaccine doses were administered 2 weeks apart. Prior to formulating the vaccine, P. yoelii 17X pRBCs and nRBCs were either treated with mPEG (treated) or PBS (untreated) and freeze thawed 6-8 times to lyse the RBC membranes. One month following administration of the final vaccine dose, spleens were removed, processed and splenocytes (4×10⁵/well) were incubated with P. yoelii 17X pRBCs (5×10⁵/well), nRBCs (5×10⁵/well), media or ConA for 72 hours at 37 °C/5% CO2. A portion of the splenocyte culture supernatants were removed after 50-54 hours of incubation and frozen at −80 °C. These supernatants were applied into the mouse Th1/Th2/Th17 BD Cytometric Array according to manufacturer's instructions to quantify cytokines.

FIG. 6: Treatment of human red blood cells (normal or parasitised) with mPEG-cyanuric chloride reduces RHD/CE expression. Normal human red blood cells (nRBCs) or purified P. falciparum parasitised red blood cells (pRBCs) derived from in vitro culture were treated with mPEG according to a standard protocol. Control RBCs were treated with phosphate buffered saline (PBS). Following treatment, RBCs were washed and stained with a fluorophore conjugated RHD/CE antibody. Using flow cytometry, RHD/CE expression on mPEG treated and PBS treated cells were compared.

FIG. 7: mPEG-cyanuric chloride treatment of P. falciparum pRBCs in a whole blood-stage parasite malaria vaccine significantly reduces production of antibodies that recognise the surface of normal human red blood cells in inbred mice. BALB/c mice were vaccinated sub-cutaneously (s.c.) with 3 doses of 10⁷ P. falciparum parasitised red blood cells (pRBCs) or normal human red blood cells (nRBCs) formulated with PHAD liposomes (1). Vaccine doses were either prepared fresh or flash frozen prior to administration and were administered 2 weeks apart. Prior to formulating the vaccine, P. falciparum pRBCs and nRBCs were either treated with mPEG (treated) or PBS (untreated) and freeze thawed 6-8 times to lyse the RBC membranes. One month following administration of the final vaccine dose, serum was collected from the mice. The induction of antibodies recognising the surface of human red blood cells was assessed using serum from vaccinated mice in a flow-cytometry-based serum binding assay.

FIG. 8: mPEG-cyanuric chloride treatment of P. falciparum pRBCs in a whole blood-stage parasite malaria vaccine significantly reduces production of antibodies that recognise the surface of normal human red blood cells in outbred mice. Outbred Swiss mice were vaccinated sub-cutaneously (s.c.) with 3 doses of 10⁷ P. falciparum parasitised red blood cells (pRBCs) or normal human red blood cells (nRBCs) formulated with PHAD liposomes (1). Vaccine doses were either prepared fresh or flash frozen prior to administration and were administered 2 weeks apart. Prior to formulating the vaccine, P. falciparum pRBCs and nRBCs were either treated with mPEG (treated) or PBS (untreated) and freeze thawed 6-8 times to lyse the RBC membranes. One month following administration of the final vaccine dose, serum was collected from the mice. The induction of antibodies recognising the surface of human red blood cells was assessed using serum from vaccinated mice in a flow-cytometry-based serum binding assay.

### DETAILED DESCRIPTION

### General Techniques and Definitions

Unless specifically defined otherwise, all technical and scientific terms used herein shall be taken to have the same meaning as commonly understood by one of ordinary skill in the art (e.g. in immunology, molecular biology, immunohistochemistry, biochemistry, genomics and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an antigen” is a reference to one or more antigens.

The term “and/or”, e.g., “X and/or Y” shall be understood to mean either “X and Y” or “X or Y” and shall be taken to provide explicit support for both meanings or for either meaning.

As used herein, the terms “approximately” and “about” refer to tolerances or variances associated with numerical values recited herein (e.g., ±0.1%, 0.5%, 1.0%, 5.0% or 10%). The extent of such tolerances and variances are well understood by persons skilled in the art.

Typically, such tolerances and variances do not compromise the structure, function and/or implementation of the compositions and methods described herein.

Throughout this specification, the word “comprise’ or variations such as “comprises” or “comprising” will be understood to imply the inclusion of a stated element, integer or step, or group of elements, integers or steps, but not the exclusion of any other element, integer or step, or group of elements, integers or steps.

By “consisting essentially of”, in the context of an amino acid sequence, is meant the recited amino acid sequence together with an additional one, two or three amino acids at the N- or C-terminus. Moreover, in the context of a nucleotide sequence, the term “consisting essentially of”, is meant the recited nucleotide sequence together with an additional one, two or three nucleic acid residues or bases at the 5′ end or 3′ end thereof.

All computer programs, algorithms, patent and scientific literature referred to herein is incorporated in their entirety herein by reference.

### Immunogenic Compositions

The present inventors sought to develop a whole parasite vaccine for erythrocytic parasites, such as malaria, that provided protection against infection by such organisms, whilst also minimising the production of alloantibodies to red blood cell antigens in patients. They demonstrate herein proof-of-principle that a whole parasite vaccine including erythrocytes treated with an antigen masking agent, such as methoxypolyethylene glycol (mPEG), minimises the immunogenicity of the erythrocytes within the vaccine composition, whilst also not altering the immunogenicity of the vaccine composition in relation to the erythrocytic parasite therein. Surprisingly, whole parasite vaccine compositions containing erythrocytes treated with mPEG described herein further demonstrated significantly better clinical results in a mouse model of malarial infection, suggesting that such compositions also demonstrate superior protection against the development of disease following infection with erythrocytic parasites. Such whole parasite vaccine compositions as described herein also advantageously demonstrate similar levels of toxicity as control vaccine compositions that do not include an antigen masking agent.

A major advantage of whole parasite vaccines is that every antigen in the organism is represented in the vaccine, limiting the effects of antigenic variation and polymorphism.

Accordingly, in a broad form the present disclosure provides an immunogenic composition comprising:
a plurality of erythrocytes and/or components or fragments thereof;
a plurality of erythrocytic organisms; and
an antigen masking agent conjugated, linked or otherwise associated with the erythrocytes and/or components or fragments thereof.

As used herein, the term “immunogenic” will be understood to mean that the composition induces, elicits or generates an immune response, such as an immune response specific for one or more antigens of the erythrocytic organism. In this regard, the antigen masking agent is suitably not or not substantially bound, conjugated, linked or otherwise associated with the erythrocytic organisms of the immunogenic composition. As such, the antigen masking agent suitably does not substantially mask one or more antigens of the erythrocytic organism. Correspondingly, the term “non-immunogenic”, as used herein, means incapable of, or substantially incapable of, provoking an immune response in the body of a human and other animal. As used herein, the term “low immunogenicity” refers to an immune response that is substantially weaker or lower than the immune response elicited by a reference molecule that is considered immunogenic in nature, for example, when injected subcutaneously or intramuscularly into a patient.

Suitably, the immune response elicited by the immunogenic composition described herein is a protective immune response. By “protective immune response” is meant an immune response that is sufficient to prevent or at least reduce the severity or symptoms of an infection with the erythrocytic organism, such as a malaria parasite, in a subject. As used herein, “elicits an immune response” or “induces an immune response” indicates the ability or potential of the immunogenic composition to elicit or generate an immune response to the erythrocytic organism, upon administration to the subject. As used herein, “immunize” and “immunization” refer to administering the immunogenic composition to elicit or potentiate a protective immune response to the erythrocytic organism.

As used herein, the term “subject” refers to any animal, for example, a mammalian animal, including, but not limited to humans, non-human primates, livestock (e.g. sheep, horses, cattle, pigs, goats, donkeys, deer), companion animals (e.g. pets such as dogs and cats), laboratory test animals (e.g. mice, rabbits, rats, guinea pigs), performance animals (e.g. racehorses, camels, greyhounds) or captive wild animals. Suitably, the term “subject” refers to any mammal capable of infection by the erythrocytic organism or parasite described herein, such as a malaria parasite. In particular examples, the subject is a human. Typically, the terms “subject” and “patient” are used interchangeably, particularly in reference to a human subject.

According to alternative examples, the subject is a non-human mammal. In this regard, the immunogenic composition may be considered a veterinary composition for use in the treatment, amelioration and/or prevention of an infection with the erythrocytic organism in a domesticated mammal. For such examples, the subject is suitably a dog or a bovine.

As used herein, “bovines” are members of the mammalian sub-family Bovinae and include cattle, buffalo, bison and yaks. Cattle include all breeds and sub-species of the genus Bos, including Bos indicus and Bos taurus and hybrids thereof.

As used herein, the term “canine” refers to an animal that is a member of the Canidae family, including dingo, wolf, jackal, fox, coyote, and the domestic dog. Dogs include all breeds and sub-species of the species Canis lupas familiaris or Canis familiaris.

The terms “red blood cells”, “RBCs” or “erythrocytes” as used herein refer to blood cells, inclusive of precursor or immature blood cells, capable of transporting oxygen. Erythrocytes for use in the present disclosure may be derived from any suitable source of whole blood using any suitable or standard preparation protocol. In the present disclosure, the erythrocytes may be obtained from a source of donor blood which is intended for use, for example, as an immunogenic composition. Suitably, the erythrocytes are obtained from a source or species that is currently infected or is capable of being infected, such as under in vitro culture conditions, with the erythrocytic organism. In particular examples, the erythrocytes are obtained from a mammalian species that is currently infected or is capable of being infected with a Plasmodium sp., such as those described herein. The erythrocytes of the immunogenic composition are therefore generally obtained or derived from a human donor. In other examples, such as those related to Babesia sp., the erythrocytes of the immunogenic composition are obtained or derived from a canine donor or a bovine donor.

### Antigen Masking Agents

The presence of the antigen masking agent in the immunogenic composition described herein is intended to substantially mask one or more antigens of the erythrocytes therein by being linked, conjugated or associated therewith. Suitably, the antigen masking agent demonstrates low immunogenicity or is non-immunogenic or substantially non-immunogenic.

Suitably, one or more antigens of the erythrocytes (e.g., one or more red blood cell antigens) are substantially masked by the antigen masking agent. Masking of such antigens involves the modification, shielding or hiding of these antigens so that they are no longer accessible to or recognized by the immune system of the subject receiving the immunogenic composition. This arrangement should advantageously avoid or reduce the likelihood of the production of allotypic anti-RBC antibodies in a subject receiving the immunogenic composition and by extension the development of an auto-immune haemolytic condition if the subject is administered non-matched RBCs in the future.

The term “red blood cell antigen” refers to any antigen found on the surface of a red blood cell or expressed by a red blood cell. RBC antigens may be grouped according to the moieties on which they are found. For example, the “K” and “k” RBC antigens are found on the “Kell” glycoprotein, and belong to the “Kell group” of RBC antigens. Groups of antigens may be referred to as a “blood group” or “blood group system”, for example, the “Kell blood group”. Each blood group may contain several different antigens, sometimes as many as 50 or more antigens. RBCs from one individual may be positive or negative for different antigens within the same blood group.

RBC antigens may be carbohydrates, for example the ABO group antigens, or may be proteins, for example, the Rhesus (Rh) group antigens. When a patient is exposed to an RBC antigen, they may produce antibodies against that antigen, such as alloantibodies of the IgG subtype or IgM subtype. Binding of patient antibodies to RBC antigens in vivo can result in destruction or haemolysis of the RBC. RBC antigens against which clinically significant patient antibodies may occur include the Ab group, ABO group, Cromer group, Diego group, Duffy group, Gerbich group, GLOB group, Indian group, Kell group, Kidd group, Knops group, Lewis group, Lutheran group, LW group, MNS group, P1 group, Rh group, XK group, Xg group, and the Yt group.

Accordingly, the antigen masking agent suitably reduces the immunogenicity of the erythrocytes and/or components or fragments thereof (e.g., reduces the likelihood of the production of allotypic anti-RBC antibodies in a subject receiving the immunogenic composition) relative to an untreated erythrocyte control (or an immunogenic composition comprising such untreated erythrocytes). This reduction in immunogenicity with respect to the treated erythrocytes and/or components or fragments thereof may be assessed by any means known in the art. Certain in vitro assays, known to those skilled in the art, may be carried out to assess immunogenicity of a treated red blood cell relative to an untreated control red blood cell. In vitro assays include, but are not limited to, ABO reactivity agglutination, red blood cell aggregation, reactivity to minor antigens, an ELISA assay to measure direct binding of alloantibodies to the treated erythrocytes, and analysis of bound labelled or fluorescent antibody on treated erythrocytes to assess levels of modification by the immune or antigen masking agent (see, e.g., Example 2).

Red blood cell antigens are considered to be substantially masked when the erythrocytes treated with the antigen masking agent are demonstrated to have significantly reduced reactivity toward antibodies that bind specific red blood cell antigens when compared to the binding of an untreated red blood cell. This reduced immunogenicity can be readily measured using in vitro antibody binding assays or in vitro measurements of the amount of modification of the red blood cells, as described above.

By way of example, an immunogenic composition comprising Rh positive (i.e., has the D antigen) erythrocytes treated with an antigen masking agent demonstrates reduced binding of an anti-D antibody to the treated red blood cells relative to an untreated Rh positive red blood cell control, such as measured by flow cytometry or an ELISA assay. In particular examples, in vitro binding of an anti-D antibody to the treated erythrocytes is reduced by at least 75% (e.g., at least 75%, 80%, 85%, 90%, 95%, 96%, 97%, 98%, 99% or any range therein), more particularly at least 90%, even more particularly at least 95%, or yet even more particularly more than or at least 99% compared to the untreated erythrocytes.

Similarly, an immunogenic composition comprising erythrocytes treated with an antigen masking agent demonstrates reduced binding of an anti-TER119 antibody to the treated red blood cells relative to an untreated red blood cell control, such as measured by flow cytometry or an ELISA assay. In particular examples, in vitro binding of an anti-TER119 antibody to the treated erythrocytes is reduced by at least 75% (e.g., at least 75%, 80%, 85%, 90%, 95%, 96%, 97%, 98%, 99% or any range therein), more particularly at least 90%, even more particularly at least 95%, or yet even more particularly more than or at least 99% compared to the untreated erythrocytes.

The immunogenicity of minor antigens, which are generally implicated in alloimmunization, can also be tested in vitro. An exemplary system known in the art rates the reaction on a scale of 0-4+, 0 being no reaction, 4+ being the highest level of agglutination (see, e.g., Walker et al., AABB Technical Manual, 10th Ed., pp 528-537 (1990)). Minor antigens that have been implicated in alloantibody production include Jka, E, K, Bg, Lua, P1, D, Sda, Fya, M, Yka, A1, Lea, Kpa, C, e, and I (Heddle et al., Brit. J. Hemat. 91:1000-5 (1995)). In particular examples, the immunogenic composition provided herein demonstrates in vitro reactivity in this assay for anti-D, anti-Jka, anti-E, anti-C, anti-e, or anti-K of less than or equal to 2+, less than or equal to 1+, or 0 on this rating scale of 0-4+.

The erythrocytes treated with the antigen masking agent suitably have reduced immunogenicity or are less immunogenic. As used herein, the terms “reduced immunogenicity” and “less immunogenic” means that when the immunogenic composition comprising these treated erythrocytes is administered to a subject (e.g., an antigen mismatched subject) in need thereof, the treated erythrocytes with reduced immunogenicity (i.e., reduced RBC antigen immunogenicity) cause or elicit a reduced immune response compared to the same erythrocytes (e.g., same RBC antigens) that have not been treated with an antigen masking agent. The reduced immune response includes, but is not limited to, a reduction in alloantibodies, or a level of production thereof.

As used herein, the term “alloantibody” refers to an antibody that specifically binds a red blood cell antigen that is not present on the subject's own red blood cells. The alloantibodies may therefore be referred to as anti-red blood cell antigen alloantibodies. Alloantibodies are distinguishable from “autoantibodies”, which refers to an antibody that specifically binds an antigen present on the subject's own red blood cells. Alloantibodies may be detected by any methods known in the art, such as those described herein. For an alloantibody to develop, an individual must be exposed to a non-self RBC antigen and have an HLA-binding motif capable of presenting a portion of the non-self antigen (Tormey & Hendrickson, 2019).

In view of the foregoing, administration of the immunogenic composition provided herein to an antigen (e.g., ABO) mismatched recipient (e.g., treated type A erythrocytes administered to a type B recipient) suitably does not result in the production of alloantibodies or results in the reduced production of alloantibodies compared to the immune reaction produced in a similarly antigen mismatched recipient after administration of a control immunogenic composition that comprises erythrocytes that have not been treated with the antigen masking agent.

The terms, “lower”, “reduced” and “decreased”, as used herein refer to a lower amount or level of alloantibody production in a subject administered the immunogenic composition provided herein, when compared to a control immunogenic composition (e.g., a control immunogenic composition comprising a plurality of erythrocytes that have not been contacted or treated with the antigen masking agent). The level or amount of alloantibodies in the subject may be relative or absolute (i.e., relatively or absolutely lower, reduced or decreased). In some examples, the level or amount of alloantibodies in the subject is lower, reduced or decreased if its level is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of alloantibodies in the subject when administered a control immunogenic composition in which the erythrocytes that have not been contacted with the antigen masking agent.

In particular examples, one or more antigens, such as one or more red blood cell antigens, are substantially masked so that transfusion or administration of the treated erythrocytes into an antigen mismatched subject would result in a reduced immune reaction (e.g., reduced production of alloantibodies) compared to the immune reaction as a result of transfusion or administration of control untreated erythrocytes into the antigen mismatched subject.

Antigen masking agents may include a non-immunogenic group and/or a coupling group for covalently binding to erythrocytes in order to mask antigens thereon. Exemplary antigen binding agents capable of blocking recognition of antigens on an erythrocyte surface when covalently attached thereto include, but are not limited to, polyalkylene glycols (such as PEG, polypropylene glycol, mixed polypropylene-polyethylene glycols), polyalkylene glycol derivatives (such as methoxypolyethylene glycol, mPEG), polysaccharides (such as dextrans, cellulosics, Ficoll, and arabinogalactan), hydrophilic, synthetic polymers, such as hyperbranched polyglycerols (HPG) (see, e.g., Rossi et al., Biomaterials 2010; 31:4167-78) and polyurethanes, and proteins, such as polydopamine (see e.g., Wang et al., Chem Sci 2014, 5:3463). In some examples, the antigen masking agent is or comprises mPEG. More particularly, the antigen masking agent suitably is or comprises cyanuric chloride-conjugated mPEG. In other examples, the antigen masking agent is or comprises a hyperbranched polyglycerol. In certain examples, the antigen masking agent is or comprises a polydopamine.

According to particular examples, the antigen masking agent is or comprises a polyethylene glycol or a derivative thereof. Such polyethylene glycol compounds may have the general formula Cp-(OCH₂CH₂)ₙ—OH wherein n is greater than or equal to 3 and Cp represents a coupling group which reacts with terminal thiol or amine groups on a red blood cell surface to covalently link the non-immunogenic group to the red blood cell. PEG derivatives in which the end groups are modified include, but are not limited to, PEG ethers (e.g., Cp-(OCH₂CH₂)ₙ—OR, such as Cp-(OHC₂CH₂)ₙ—OCH₃ (MPEG)), PEG esters (e.g. Cp-(OCH₂CH₂)ₙμ-OOCR, such as Cp-OCH₂CH₂)ₙ OOC(CH₂)₁₄CH₃), PEG amides (e.g. Cp (OCH₂CH₂)ₙ—OOC(CH₂)₇CONHR), PEG amines (e.g. Cp-(OCH₂CH₂)ₙ—NH₂), PEG acids (e.g. Cp-(OCH₂CH₂)ₙ—OCH₂COOH), PEG aldehydes (e.g. Cp (OCH₂CH₂)ₙ—OCH₂CHO), and electrophilic derivatives, such as halogenated PEG (e.g. Cp-(OCH₂CH₂)ₙ—Br).

Suitably, the coupling group for linking the non-immunogenic group of the antigen masking group to the erythrocytes comprises a reactive group which reacts with terminal thiol or amine groups on the red blood cell surface. It is contemplated, however, that the antigen masking agent described herein is not to be limited to any particular coupling agent. Examples include, but are not limited to, sulphonate esters, substituted triazines, N-hydroxysuccinimide esters, anhydrides, substituted phenyl carbonates, oxycarbonylimidazoles, maleimides, aldehydes, glyoxals, carboxylates, vinyl sulphones, epoxides, mustard, mustard equivalents, isocyanates, disulphides, acrylates, allyl ethers, silanes, cyanate esters and halogen atoms. Exemplary coupling groups include, but are not limited to, aziridine derivatives, mono or bis-(mesylethyl)amine groups, mono mesylethylsulfide groups, mono- or bis-tosylethylamine groups, and mono-tosylethylsulfide groups, 2,2,2-trifluoroethanesulphonate, pentafluorobenzenesulphonate, fluorosulphonate, 2,4,5-trifluorobenzenesulphonate, 2,4-difluorobenzenesulphonate, 2-chloro-4-fluorobenzenesulphonate, 3-chloro-4-fluorobenzenesulphonate, 4-amino-3-chlorobenzenesulphonate, 4-amino-3-fluorobenzenesulphonate, o-trifluoromethylbenzenesulphonate, m-trifluoromethylbenzenesulphonate, 2-trifluoromethoxybenzenesulphonate, 4-trifluoromethoxybenzenesulphonate, 5-fluoro-2-methylbenzenesulphonate, 4,6-dichlorotriazine, 6-chlorotriazine, N-hydroxysuccinimidyl succinate, N-hydroxysuccinimidyl glutarate, N-hydroxysuccinimidyl succinamide, N-hydroxysuccinimidylalkanedioicamides, N-hydroxysuccinimidyl derivatives of carboxymethylated polymers, N-hydroxysuccinimidyl esters of amino acids, succinimidylcarbonate, succinate mixed anhydride, succinic anhydride, 2,4,5-trichlorophenol, trichlorophenyl carbonate, nitrophenyl carbonate, 4-nitrophenol, cyanuric chloride, maleimide, N-substituted maleimide, acetaldehyde, propionaldehyde and chemically equivalent sulfur analogs, glyoxal, phenylglyoxal, acrylate, methacrylate, iodide, bromide and chloride. In particular examples, the coupling group of the antigen masking agent is or comprises cyanuric chloride.

### Erythrocytic Organisms

It is contemplated that the erythrocytic organisms may be any as are known in the art. Suitably, the erythrocytic organisms are intra-erythrocytic organisms or intra-erythrocytic parasites.

In some examples, the erythrocytic organisms are apicomplexan or an apicomplexan parasite. Accordingly, the immunogenic composition can include blood-stage intra-erythrocytic parasites, such as merozoites, schizonts, rings or trophozoites, although without limitation thereto. For example, the blood-stage intra-erythrocytic parasites may be purified merozoites or a mixture of isolated merozoites and other blood-stage intra-erythrocytic parasites, such as schizonts, rings and/or trophozoites.

For the purposes of this disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

In some examples, the erythrocytic organisms are attenuated, inactivated and/or killed. Suitably, the erythrocytic organisms are attenuated, inactivated and/or killed whilst in their corresponding erythrocytes. In alternative examples, the erythrocytic organisms are attenuated, inactivated and/or killed prior to their addition to the corresponding erythrocytes.

In certain examples, the erythrocytic organism is attenuated. The term “attenuate” means to modify the erythrocytic organisms in a way that they becomes less virulent or pathogenic than they were prior to treatment. More particularly, this typically means that the erythrocytic organism exhibits a substantially reduced ability to cause a clinical disease while still being able to replicate in the host. Methods of attenuating parasites are known in the art and described, for example, in Good et al., 2013 (Journal of Clinical Investigation). With respect to attenuated erythrocytic organisms, these are suitably included intact in the immunogenic composition.

In particular examples, the erythrocytic organism is inactivated. The term “inactivate” means the erythrocytic organisms have been modified in a way or killed such that they are incapable of reproduction in vivo or in vitro. Various physical and chemical methods of inactivating microorganisms are known in the art, such as being irradiated (e.g., treated with UV, X-ray, electron beam or gamma radiation), heat treated, or chemically treated. In this regard, the immunogenic composition may include a lysate, a fraction or one or more components or fragments of the erythrocytic organisms described herein.

According to particular examples, the immunogenic composition is suitably a whole parasite vaccine, such as an attenuated whole parasite vaccine, an inactivated whole parasite vaccine or a killed whole parasite vaccine.

In some examples, the erythrocytes are initially infected with the erythrocytic organisms and then treated to kill, attenuate or inactivate the erythrocytic organisms associated therewith. In alternative examples, the erythrocytic organisms are killed prior to being combined or mixed with the erythrocytes, such as lysates thereof.

Suitably, the erythrocytes have been previously infected with the erythrocytic organism. In this regard, the erythrocytes may be infected with the erythrocytic organism in vitro, ex vivo and/or in vivo, such as by means or methods well known in the art.

According to particular examples, the immunogenic composition comprises a single species of the erythrocytic organism (e.g., a single Plasmodium sp.). In alternative examples, the immunogenic composition comprises two or more species (e.g., 2, 3, 4, 5 etc) of the erythrocytic organism from a single genus (e.g., 2, 3, 4 or 5 different Plasmodium spp.).

In other examples, the immunogenic composition comprises two or more species (e.g., 2, 3, 4, 5 etc) of the erythrocytic organism from two or more genera (e.g., 2, 3, 4, 5 etc) thereof. By way of example, the immunogenic composition may comprise one or more species of a first erythrocytic organism from a first genus and one or more species of a second erythrocytic organism from a second genus. In certain examples, the immunogenic comprises a first erythrocytic organism of the genus Plasmodium and a second erythrocytic organism of the genus Babesia. In such examples, the subject is suitably a human. In alternative examples, the immunogenic comprises a first erythrocytic organism of the genus Babesia and a second erythrocytic organism of the genus Anaplasma. For such examples, the subject is suitably a bovine animal. In further examples, the immunogenic comprises a first erythrocytic organism of the genus Babesia and a second erythrocytic organism of the genus Theleria. Again, for such examples, the subject is suitably a bovine animal.

In some examples, the erythrocytic organism is selected from the group consisting of a Plasmodium sp., a Babesia sp., an Anaplasma sp., an Ehrlichia sp., a Trypanosoma sp., a Theileria sp., a Hepatozoon sp., a Mycoplasma sp., a Bartonella sp. and any combination thereof. Exemplary Plasmodium species include P. falciparum, P. vivax, P. malariae, P. ovale, P. knowlesi, P. berghi, P. yoelii, P. chabaudi and P. vinckei. Exemplary Babesia species include B. bigemina, B. bovis, B. caballi, B. canis, B. divergens, B. microti, and B. motasi. Exemplary Anaplasma species include Anaplasma platys, Anaplasma phagocytophila, Anaplasma marginale, and Anaplasma centrale. Exemplary Ehrlichia species include Ehrlichia canis. Exemplary Trypanosoma species include Trypanosoma congolense, Trypanosoma evansi and Trypanosoma cruzi. Exemplary Theileria species include Theileria orientalis, Theileria buffeli, Theileria annulata and Theileria parva. Exemplary Hepatozoon species include Hepatozoon canis and Hepatozoon americanum. Exemplary Mycoplasma species include Mycoplasma haemocanis. Exemplary Bartonella species include Bartonella vinsonii subsp. Berkhoffii, Bartonella henselae, Bartonella bovis and Bartonella chomelii.

According to certain examples, the erythrocytic organism is or comprises a Plasmodium sp. As used herein, “Plasmodium”, “malaria” or “malarial” parasites are any pathogenic protozoa of the genus “Plasmodium” and are not limited to a particular species, subspecies, strain, variant, isolate, or the like. The genus “Plasmodium” includes pathogenic species such as P. falciparum, P. malariae, P. vivax, P. ovale and P. knowlesi. In particular examples, the erythrocytic organism is or comprises P. falciparum. In other examples, the erythrocytic organism is or comprises P. malariae. In some examples, the erythrocytic organism is or comprises P. vivax. In various examples, the erythrocytic organism is or comprises P. ovale. In certain examples, the erythrocytic organism is or comprises P. knowlesi. In other examples, the erythrocytic organism is or comprises P. yoelii.

Accordingly, in a particular form, the present disclosure provides a method of treating, ameliorating or preventing malaria in a subject, said method including the step of administering the immunogenic composition disclosed herein to the subject to thereby prevent or inhibit a malarial infection or treat or ameliorate an existing malarial infection in the subject.

As used herein, “malaria” includes all forms of the disease caused by protozoal parasites of the genus Plasmodium, such as those hereinbefore described. In examples relevant to malaria in humans, the causative malaria species are typically P. falciparum, P. malariae, P. vivax, P. ovale and P. knowlesi. Suitably, for treatment of humans, the immunogenic composition and method of prophylactic or therapeutic treatment of malaria comprises human erythrocytes infected with said Plasmodium parasites, such as one or more of P. falciparum, P. malariae, P. vivax, P. ovale and P. knowlesi and coated with an antigen masking agent, such as mPEG. In one particular example, the immunogenic composition and method of prophylactic or therapeutic treatment of malaria in humans comprises human erythrocytes that have been infected with Plasmodium parasites, such as P. falciparum, P. malariae, P. vivax, P. ovale and P. knowlesi parasites, which have been attenuated, inactivated or killed, and coated or treated with an antigen masking agent, such as mPEG.

In particular examples, the erythrocytic organism is or comprises a Babesia sp. For such examples, the subject is suitably a human, a canine animal or a bovine animal. As used herein “Babesia” parasites are any pathogenic protists of the genus “Babesia”. The genus “Babesia” includes pathogenic species such as Babesia bovis, Babesia canis, Babesia bigemina, Babesia divergens, Babesia microti, Babesia caballi, Babesia duncani, Babesia venatorum, Babesia ovis, Babesia ovata, Babesia occultans, Babesia vogeli, Babesia gibsoni and Babesia motasi although without limitation thereto. In one example, the erythrocytic organism is or comprises Babesia divergens. As used herein, “babesiosis” includes all forms of the disease caused by parasitic organisms of the genus Babesia, such as those hereinbefore described.

Therefore, in a particular form, the present disclosure provides a method of treating, ameliorating or preventing babesiosis in a subject, said method including the step of administering the immunogenic composition disclosed herein to the subject to thereby prevent or inhibit or ameliorate a babesial infection or treat an existing babesial infection in the subject.

In other examples, the erythrocytic organism is or comprises a Theileria sp. For such examples, the subject is suitably a bovine animal. The term “Theileria” refers to a genus of apicomplexan parasite that includes Theileria orientalis, Theileria buffeli, Theileria annulata and Theileria parva, although without limitation thereto.

As such, in a particular form, the present disclosure provides a method of treating, ameliorating or preventing theileriosis in a subject, said method including the step of administering the immunogenic composition disclosed herein to the subject to thereby prevent or inhibit a theilerial infection or treat or ameliorate an existing theilerial infection in the subject.

The immunogenic composition described herein may, upon administration to the subject, immunize against infection by heterologous isolates, strains and/or species of the erythrocytic organism. By “heterologous” pathogens means related pathogens that maybe different strains or variants of a same or related species. The skilled artisan will further appreciate that the immunogenic composition described herein may, upon administration to the subject, immunize against infection by the erythrocytic organism itself and optionally one or more heterologous isolates, strains and/or species thereof.

Accordingly, in some examples, the immunogenic composition provides heterologous protection against an infection, disease, disorder or condition associated with one or more other isolates, strains and/or species of Plasmodium. For example, administration of an immunogenic composition comprising erythrocytes, such as human erythrocytes, and blood-stage Plasmodium falciparum: parasites, such as those that are attenuated, inactivated or killed, to the subject, can provide heterologous protection against an infection, disease, disorder or condition associated with P. malariae, P. vivax, P. ovale and/or P. knowlesi.

Suitably, the immunogenic composition and/or method of prevention, amelioration or treatment of malaria are at least partly effective against blood-stage malaria. Blood-stage malaria is a stage where the Plasmodium parasite (e.g., merozoite) enters erythrocytes. In the blood stage, the parasite matures and divides several times to produce new merozoites, which leave the red blood cells and travel within the bloodstream to invade new red blood cells. For the sufferer, blood-stage malaria is responsible for the clinical manifestations of the disease and is typically characterized by successive waves of fever arising from simultaneous waves of merozoites escaping and infecting red blood cells.

### DNA Binding Agents

In some examples, the erythrocytic organisms are chemically attenuated, such as by treatment with a DNA binding agent.

In some examples, the DNA binding agent is centanamycin or an analog or derivative thereof. Centanamycin is a rationally designed, achiral DNA binding and alkylating agent based on (+)-duocarmycin SA that lacks a stereocenter. Centanamycin binds covalently to adenine-N3 in the DNA sequence motif (A/T)AAA.

In other examples, the DNA binding agent is tafuramycin A or an analog or derivative thereof.

Tafuramycin A is a rationally designed, DNA binding and alkylating agent based on duocarmycins that comprises a stereocenter.

By “centanamycin or tafuramycin A analogs or derivatives” is meant any molecule structurally related to centanamycin or, tafuramycin A which exhibits binding to AT-containing nucleotide sequences to thereby induce DNA damage.

Centanamycin, tafuramycin A, analogs or derivatives inclusive of non-chiral, chiral and racemic analogs and derivatives of duocarmycin and CC-1065, non-chiral, chiral and racemic isomers, salts or solvates thereof are also described in WO2002/030894, WO2008/050140, WO2009/064908, Howard et al., 2002 and Purnell et al., 2006, Chavda et al., 2010 and U.S. Pat. No. 6,660,742, which are incorporated by reference herein. Reference is particularly made to seco-iso-cyclopropylfurano[2,3-e]indoline-TMI (TH-III-149 or tafuramycin A) and seco-cyclopropyltetrahydrofurano[2,3-f]quinoline-TMI (TH-III-151 or tafuramycin B) analogs of CC-1065 and the duocarmycins as described in Howard et al., 2002, supra and Purnell et al., 2006, supra.

Achiral seco-hydroxy-aza-CBI-TMI, a seco-cyclopropylpyrido[e]indolone (CPyI) compound, is an example of an analog of centanamcyin as described in Chavda et al., 2010, supra. Racemic and chiral 5-methylfuran analogs of tafuramycin A are described in Purnell et al., 2006, supra.

The chemical formula of tafuramycin A is provided below:

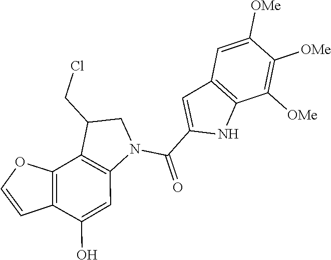 [SMILES: COc1cc2cc(C(=O)N3CC(CCl)c4c3cc(O)c3ccoc43)[nH]c2c(OC)c1OC]

The chemical formula of centanamycin is provided below:

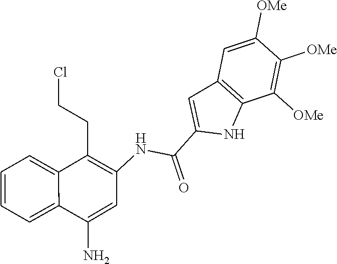 [SMILES: COc1cc2cc(C(=O)Nc3cc(N)c4ccccc4c3CCCl)[nH]c2c(OC)c1OC]

Non-limiting concentrations of the DNA binding agent, including tafuramycin A, centanamycin, or analogs or derivatives thereof, for treatment of red blood cells infected with the erythrocytic organisms described herein or the erythrocytic organisms prior to their infection of or addition to the red blood cells, are in the range of about 0.1 to 100 M. Suitably, the concentration is in the range of about 0.5-50 μM, more preferably in the range about 1-40 M or even more preferably in the range of about 2-20 μM. Particular concentrations include about 0.5 μM, 1.0 μM, 1.5 μM, 2.0 μM, 3 μM, 4 μM, 5.0 μM, 6.0 μM, 7.0 μM, 8.0 μM, 9.0 μM, 10 μM, 11 μM, 12 μM, 13 μM, 14 μM, 15 μM, 16 M, 17 μM, 18 M, 19 μM, 20 μM, 25 μM and 30 M, or in any range therebetween.

Treatment duration of the erythrocytic organisms with the DNA binding agent may be in the range of about 1 minute to about 12 hours (e.g., about 0.1, 0.2, 03, 0.4, 0.5, 0.6, 0.7, 0.8, 0.9, 1.0, 1.5, 2, 2.5, 3.0, 3.5, 4.0, 4.5, 5.0, 6.0, 7.0, 8.0, 9.0, 10.0, 11.0, 12.0 hours or any range therein), more particularly 10 minutes to 4 hours or even more particularly about 0.1, 0.2, 03, 0.4, 0.5, 0.6, 0.7, 0.8, 0.9, 1.0, 1.5 or 2 hours, or any range therebetween.

The DNA binding agent-treated erythrocytic organisms may be used to infect a population of red blood cells or erythrocytes, which are then used to prepare the immunogenic composition. Alternatively, red blood cells infected with erythrocytic organisms may be treated with the DNA binding agent and then used to prepare the immunogenic composition.

Suitably, the erythrocytes in the immunogenic composition may be administered as intact cells and/or as a lysate (e.g., lysed cells). According to some examples, the erythrocytes in the immunogenic composition are substantially included as intact cells (e.g., greater than 75%, 80%, 85%, 90%, 95% or 99% of the erythrocytes in the immunogenic composition are intact cells). In alternative examples, the erythrocytes in the immunogenic composition are substantially included as a lysate or lysed cells (e.g., greater than 75%, 80%, 85%, 90%, 95% or 99% of the erythrocytes in the immunogenic composition are lysed cells). In such examples, the erythrocytes may be lysed by any means known in the art, such as one or more freeze-thaw cycles, mechanical force (e.g., homogenization, a cell press), a detergent and/or sonication.

It will be appreciated from the foregoing that particular examples of the present disclosure relates to in vitro treatment of isolated or purified blood-stage intra-erythrocytic parasites, such as merozoites or red blood cells infected with blood-stage parasites (e.g., merozoites, schizonts, rings or trophozoites, although without limitation thereto), with a DNA binding agent (e.g., centanamycin, tafuramycin A or an analog or derivative of centanamycin or tafuramycin A). This treatment is effective to chemically attenuate the blood-stage intra-erythrocytic parasites (e.g., merozoites, schizonts, rings or trophozoites, although without limitation thereto) without killing the parasite, such as by inhibiting parasite replication. Typically, the attenuated blood-stage intra-erythrocytic parasites are not capable of proliferation, or are capable of only limited proliferation, following attenuation by treatment with the DNA binding agent.

Suitably, the dose of the erythrocytic organisms (i.e., such as in infected erythrocytes or RBCs) in the immunogenic composition is capable of eliciting an immune response to subsequent infection by said erythrocytic organism. In some examples, the immune response is characterised by inducing a T cell response and/or a B cell response to produce detectable levels of antibodies. Suitably, the immunogenic composition immunizes the subject to prevent, inhibit or otherwise protect the subject against subsequent infection with the erythrocytic organism.

In some examples, a single dose of the immunogenic composition prevents, inhibits or otherwise protects the animal against subsequent infection with the erythrocytic organism. In other examples, two or more doses (e.g., 2, 3, 4, 5 etc doses) of the immunogenic composition prevents, inhibits or otherwise protects the animal against subsequent infection with the erythrocytic organism. In one particular example, two doses of the immunogenic composition prevents, inhibits or otherwise protects the animal against subsequent infection with the erythrocytic organism. In another specific example, three doses of the immunogenic composition prevents, inhibits or otherwise protects the animal against subsequent infection with the erythrocytic organism.

A typical dose of infected erythrocytes or parasitized RBCs (pRBCs) in the immunogenic composition can be, or equivalent to, about 10² to about 10¹² pRBCs. In some examples, the immunogenic composition comprises a dose of infected erythrocytes or pRBCs suitable for administration to the subject that is no more than, or equivalent to no more than, about 10¹¹ pRBCs, such as including 10¹⁰, 10⁹, 10⁸, 10⁷, 10⁶, 10⁵, 10⁴, 10³, 10², or 10¹ pRBCs or any range between any of these values.

A typical dose of the erythrocytic organism suitable for administration to the subject is in the range of about 10² to about 10¹² erythrocytic organisms, such as including about 10², 10³, 10⁴, 10⁵, 10⁶, 10⁷, 10⁸, 10⁹, 10¹⁰, 10¹¹, or 10¹² erythrocytic organisms or any range between any of these values.

### Formulation

Suitably, the immunogenic composition comprises erythrocytes that are lysed and/or are intact. Accordingly, in examples where the erythrocytes have been lysed, the immunogenic composition may include one or more erythrocyte or RBC components, including cell membranes and fragments, membrane proteins, cytosolic components and soluble proteins thereof. In some examples, the erythrocytes of the immunogenic composition are lysed or substantially lysed. In alternative examples, the erythrocytes of the immunogenic composition are intact or substantially intact. In further examples, the immunogenic composition comprises a mixture of lysed and intact erythrocytes.

In some examples, the immunogenic composition does not include or is substantially free of an adjuvant. For such examples, the immunogenic composition suitably includes erythrocytes that are intact and contain erythrocytic organisms that have been attenuated, such as chemically attenuated with a DNA-binding agent. Furthermore, such examples may or may not be provided in particulate form, such as in the form of a lipid vesicle, a liposome or a lipid nanoparticle (LNP).

By “substantially free of an adjuvant” means that the immunogenic composition suitably contains less than 1.0% by weight, more particularly less than 0.1% by weight, even more particularly less than 0.01% by weight, or yet even more particularly less than 0.001% by weight of an adjuvant, such as those described herein.

In alternative examples, the immunogenic compositions disclosed herein may further comprise an adjuvant. For such examples, the immunogenic composition suitably includes erythrocytes that have been lysed and/or erythrocytic organisms that have been killed. In some examples, however, an immunogenic composition that includes erythrocytes that are intact and contains erythrocytic organisms that have been attenuated may further comprise an adjuvant.

The term “adjuvant” refers to a compound or mixture that enhances the immune response to an antigen. Adjuvants may act primarily as a delivery system, primarily as an immune modulator or have features of both. Suitable adjuvants include those suitable (e.g., have regulatory approval) for use in mammals, and more particularly humans.

Examples of known suitable delivery-system type adjuvants that can be used in mammals include, but are not limited to, calcium phosphate; squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, alum (e.g., aluminum phosphate, aluminum sulfate or aluminum hydroxide); interleukins such as interleukin 2 and interleukin 12; monokines such as interleukin 1; tumour necrosis factor; interferons such as gamma interferon; immunostimulatory DNA such as CpG DNA, combinations such as saponin-aluminium hydroxide or Quil-A aluminium hydroxide; liposomes (e.g., see International Publication WO2017/070735); ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol EMA; acrylic copolymer emulsions such as Neocryl A640 (e.g. U.S. Pat. No. 5,047,238); water in oil emulsifiers such as Montanide ISA 720; oil-in-water emulsions such as MF59 (4.3% w/v squalene, 0.5% w/v polysorbate 80 (Tween 80), 0.5% w/v sorbitan trioleate (Span 85)); poly(D,L-laetide-co-glycolide) (PLG) microparticles or nanoparticles (e.g., the Next Adjuvant or NexaVAC™ System from the NA Vaccine Institute); ballistic particles; ceramic particles; polymeric particles; poliovirus, vaccinia or animal poxvirus proteins; or mixtures thereof.

Suitably, the adjuvant is a TLR4 agonist, such as a lipid A glycolipid adjuvant or a analogue or derivative thereof.

It will be appreciated that the lipid A glycolipid adjuvant is an activator of innate immunity. The lipid A glycolipid adjuvant may target a C-type lectin expressed by one or more cells that are associated with innate immunity. An exemplary C-type lectin is macrophage inducible Ca²⁺-dependent (C-type) lectin (“Mincle”). Non-limiting examples include monophosphoryl lipid A (MPLA) adjuvants, which may be in the form of native monophosphoryl lipid A, monophosphoryl lipid A-504, synthetic monophosphoryl Hexa-acyl lipid A, 3-Deacyl (sPHAD), PHAD®, 3D-25 PHAD® and 3D (6-acyl) PHAD®, albeit without limitation thereto. In particular examples, the lipid A glycolipid adjuvant is 3D(6-acyl)-PHAD®. MPLA adjuvants have been shown to boost the immune system through the activation of the toll-like receptor 4 (TLR4), resulting in the production of proinflammatory cytokines and antigen-specific effector CD4+ and memory CD8+ T cells.

According to some examples, the erythrocytes or the immunogenic composition described herein, are provided in particulate form. A variety of particles may be used for the present disclosure, including but not limited to lipid vesicles, liposomes, micelles, lipidic particles (e.g., LNPs), ceramic/inorganic particles and polymeric particles. In some particular examples, the immunogenic composition is formulated into a lipid vesicle, and more particularly a liposome. Accordingly, in some examples, the erythrocytes or the immunogenic composition are contained in or otherwise associated with a particle, such as a lipid-based particle or a lipid-based vesicle.

In certain examples, the adjuvant comprises at least one vehicle compound or agent. The vehicle compound may be administered as single adjuvant compound, or, more particularly, in combination with one or more further adjuvant compounds.

Suitably, the vehicle compound is a liposome compound, such as a neutral adjuvant compound or formulation, an anionic adjuvant compound or formulation, cationic liposomal vaccine adjuvant, or Stealth liposomes, or JVRS-100 (cationic liposomal DNA complex), or cytokine-containing liposomes, or immunoliposomes containing antibodies to costimulatory molecules, or DRVs (immunoliposomes prepared from dehydration-rehydration vesicles), or MTP-PE liposomes, or Sendai proteoliposomes, or Sendai containing lipid matrices, or Walter Reed liposomes (liposomes containing lipid A adsorbed to aluminium hydroxid), or CAF01 (liposomes plus DDA plus TDB), or CAF04, or CAF09, or CAF10, or ASO1 (MPL plus liposome plus QS-21), or AS15 (MPL plus CpG plus QS-21 plus liposome). Moreover the vehicle compound may be formed by a virosome compound (unilamellar liposomal vehicles incorporating virus derived proteins, such as influenza haemagglutinin), e.g. IRIVs (immunopotentiating reconstituted influenza virosomes), or liposomes of lipids plus hemagglutinin. Moreover the vehicle compound may be formed by a virus-like particle (VLP) compound, e.g. Ty particles (Ty-VLPs). Moreover the vehicle compound may be formed by microparticles and/or nanoparticles, such as polymeric microparticles (PLG), or cationic microparticles, or albumin-heparin microparticles, or CRL1005 (block copolymer P1205), or peptomere nanoparticle, or CAP™ (calcium phosphate nanoparticles), or microspheres, or PODDS® (proteinoid microspheres), or nanospheres. Moreover, the vehicle compound may be formed by a protein cochleate compound, especially by stable protein phospholipid-calcium precipitates, such as BIORAL™. Moreover, the vehicle compound may be formed by polymeric particles, ballistic particles or ceramic particles. Also combinations of the different vehicle compounds are envisaged. Accordingly, in some examples, the immunogenic composition further comprises a lipid-containing adjuvant, a lipid-based adjuvant and/or a lipid-derived adjuvant.

Suitably, the immunogenic composition is formulated into a lipid vesicle. As broadly used herein, the lipid vesicle may be a liposome, minicell, multilamellar vesicle, micelle, vacuole, LNP or other vesicular structure comprising a lipid bilayer or a lipid monolayer. The erythrocytic organisms, whether intact or lysed and/or attenuated or killed may be located in the intravesicular space or may be displayed on a surface of the lipid vesicle.

The lipid vesicle suitably comprises any lipid or mixture of lipids capable of forming a lipid bilayer and/or monolayer structure. These include a phospholipids, sterols inclusive of cholesterol, cholesterol-esters and phytosterols, fatty acids and/or triglycerides. Nonlimiting examples of phospholipids include phosphatidylcholine (PC) (lecithin), phosphatidic acid, phosphatidylethanolamine (PE) (cephalin), phosphatidylglycerol (PG), phosphatidylserine (PS), phosphatidylinositol (PI) and sphingomyelin (SM) or natural or synthetic derivatives thereof. Natural derivatives include egg PC, egg PG, soy bean PC, hydrogenated soy bean PC, soy bean PG, brain PS, sphingolipids, brain SM, galactocerebroside, gangliosides, cerebrosides, cephalin, cardiolipin, and dicetylphosphate. Synthetic derivatives include 1,2-dipalmitoyl-sn-glycero-3-phosphocholine (DPPC), dimethyldioctadecylammonium bromide (DDAB), didecanoylphosphatidylcholine (DDPC), dierucoylphosphatidylcholine (DEPC), dimyristoylphosphatidylcholine (DMPC), distearoylphosphatidylcholine (DSPC), dilaurylphosphatidylcholine (DLPC), palmitoyloleoylphosphatidylcholine (POPC), palmitoylmyristoylphosphatidylcholine (PMPC), palmitoylstearoylphosphatidylcholine (PSPC), dioleoylphosphatidylcholine (DOPC), dioleoylphosphatidylethanolamine (DOPE), dilauroylphosphatidylglycerol (DLPG), distearoylphosphatidylglycerol (DSPG), dimyristoylphosphatidylglycerol (DMPG), dipalmitoylphosphatidylglycerol (DPPG), distearoylphosphatidylglycerol (DSPG), dioleoylphosphatidylglycerol (DOPG), palmitoyloleoylphosphatidylglycerol (POPG), dimyristoylphosphatidic acid (DMPA), dipalmitoylphosphatidic acid (DPPA), distearoylphosphatidic acid (DSPA), dimyristoylphosphatidylethanolamine (DMPE), dipalmitoylphosphatidylethanolamine (DPPE), dimyristoylphosphatidylserine (DMPS), dipalmitoylphosphatidylserine (DPPS), distearoylphosphatidylethanolamine (DSPE), dioleoylphosphatidylethanolamine (DOPE) dioleoylphosphatidylserine (DOPS), dipalmitoylsphingomyelin (DPSM) and distearoylsphingomyelin (DSSM). The phospholipid can also be a derivative or analogue of any of the above phospholipids.

According to various examples, the lipid vesicle is a liposome. Accordingly, the immunogenic composition may be formulated into a liposome. Such liposomes may include one or more further adjuvants as described herein. In some examples, the immunogenic composition is formulated into a liposome and further comprises a lipid A glycolipid adjuvant, such as 3D(6-acyl)-PHAD®.

In some examples of the present disclosure, the volume-weighted diameter of freshly prepared liposomes is between about 10 μm and about 100 μm (e.g., 10, 15, 20, 25, 30, 35, 40, 45, 50, 55, 60, 65, 70, 75, 80, 85, 90, 95, 100 μm or any range therein), about 20 μm and about 80 μm, and about 20 μm to about 50 μm. The volume weighted diameter of lyophilized liposomes may be between about 25 μm and about 150 μm, 40 μm and about 100 μm, or about 50 μm and 75 μm.

Suitably, the immunogenic composition is lyophilized. In some examples, the lipid vesicles (e.g., liposomes) are lyophilized, such as to assist with storage and delivery. Methods of lyophilising liposomes are known in the art, as disclosed by Zaman et al. (Scientific Reports volume 6, Article number: 39274, 2016) (the contents of which is included herein by reference). In particular examples, a lyoprotectant, such as hyaluronic acid, γ-cyclodextrin and/or trehalose is used. Accordingly, the immunogenic composition may further comprise a lyoprotectant. In such examples, the lyophilised liposomes can be resuspended in any suitable buffer prior to administration. Typically, the lyophilised liposomes are resuspended in PBS prior to administration.

In some examples, the erythrocytes and the erythrocytic organisms of the immunogenic composition, inclusive of lysates, components, fragments or fractions thereof, are contained in or otherwise associated with a liposome. In other examples, the erythrocytes and the erythrocytic organisms of the immunogenic composition, inclusive of lysates, components, fragments or fractions thereof, are contained in or otherwise associated with an LNP.

According to certain examples, the adjuvant is or comprises one or more cationic lipids, as are known in the art. Cationic lipids have been reported to have strong immune-stimulatory adjuvant effect. The cationic lipids of the present disclosure may form lipid vesicles, LNPs or liposomes that are optionally mixed with other components of the immunogenic composition (e.g., the treated erythrocytes and erythrocytic organisms) and may contain the cationic lipids alone or in combination with one or more neutral lipids and/or one or more additional charged lipids, such as those described herein. Suitable cationic lipid species include: 3-[4N—(N, -diguanidino spermidine) carbamoyl] cholesterol (BGSC); 3-[N,N-diguanidinoethyl-aminoethane)-carbamoyl]cholesterol (BGTC); N,N N2N3Tetra-methyltetrapalmitylspermine (cellfectin); N-t-butyl-N′ tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N dimethyl-I-p-ropanaminium trifluorocetate) (DOSPA); 1,3-dioleoyloxy-2-(6 carboxyspermyl)-propyl amide (DOSPER); 4-(2,3-bis-palmitoyloxy-propyl)-1-methyl-iH imidazole (DPIM) N,N,N′,N′-tetramethyl-N,N′-bis(2-hydroxyethyl)-2,3-dioleoyloxy-1,4-butanediammonium iodide) (Tfx-50); N-i-(2,3-dioleoyloxy) propyl-N,N,N-trimethyl ammonium chloride (DOTMA) or other N—(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-hydroxyethylammonium) or DORIE (DL-1,2-O-dioleoyl-3-dimethylaminopropyl-hydroxyethylammonium) (DORIE) or analogs thereof as disclosed in WO 93/03709; 1,2 dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 283,185, cholesteryl-3-carboxyl-amido-ethylenetrimethylammonium iodide, I dimethylamino-3-trimethylammonio-DL-2-propyl-cholestery carboxylate iodide, cholesteryl-3-O-carboxyamidoethyleneamine, cholesteryl-3-3-oxysuccinamido ethylenetrimethylammonium iodide, 1-dimethylamino-3-trimethylammonio-DL-2-propyl cholesteryl-3-3-oxysuccinate iodide, 2-(2-trimethylammonio)-ethylmethylamino ethyl cholesteryl-3-3-oxysuccinate iodide, 3-N—(N′,N′-dimethylaminoethane) carbamoyl cholesterol (DC-chol), and 3-N-(polyethyleneimine)-carbamoylcholesterol; 0,0′ dimyristyl-N-lysyl aspartate (DMKE); 0,O′-dimyristyl-N-lysyl-glutamate (DMKD); 1,2 dimyristyloxypropyl-3-dimethyl-hydroxy ethyl ammonium bromide (DMRIE); 1,2-dilauroyl sn-glycero-3-ethylphosphocholine (DLEPC); 1,2-dimyristoyl-sn-glycero-3 ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3 ethylphosphocholine (DSEPC); 1,2-dioleoyl-3-trimethylammoninum propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3 trimethylammonium propane (DMTAP); and sodium dodecyl sulfate (SDS). The present disclosure contemplates the use of structural variants and derivatives of the cationic lipids disclosed in this application.

Further to the above, the immunogenic composition may contain other lipids in addition to the cationic lipids. These lipids include, but are not limited to, lyso lipids of which lysophosphatidylcholine (1-oleoyl lysophosphatidylcholine) is an example, cholesterol, or neutral phospholipids including dioleoyl phosphatidyl ethanolamine (DOPE) or dioleoyl phosphatidylcholine (DOPC), as well as various lipophylic surfactants, containing polyethylene glycol moieties, of which Tween-80 and PEG-PE are examples. The immunogenic composition may also contain negatively charged lipids as well as cationic lipids so long as the net charge of the complexes formed is positive and/or the surface of the complex is positively charged. Negatively charged lipids of the present disclosure are those comprising at least one lipid species having a net negative charge at or near physiological pH or combinations of these. Suitable negatively charged lipid species include, but are not limited to, CHEMS (cholesteryl hemisuccinate), NGPE (N-glutaryl phosphatidlylethanolanine), phosphatidyl glycerol and phosphatidic acid or a similar phospholipid analog.

In one example, the immunogenic composition comprises a vehicle agent (e.g., liposomes) that comprises 1,2-dipalmitoylsn-glycero-3-phosphocholine (DPPC), dimethyldioctadecylammonium bromide (DDAB) and cholesterol. Suitably, the vehicle agent of the immunogenic composition comprises DPPC, DDAB and cholesterol in the ratio or molar ratio of between about 3:2:1 to about 9:2:1 or more particularly between about 5:2:1 to about 7:2:1. More particularly, the vehicle agent of the immunogenic composition can contain DPPC, DDAB and cholesterol in the ratio or molar ratio of about 5:2:1. In other examples, the vehicle agent of the immunogenic composition comprises DPPC, DDAB and cholesterol in the ratio or molar ratio of about 7:2:1.

Methods for producing liposomes to be used in the production of the immunogenic composition of the present disclosure are known to those of ordinary skill in the art. A review of methodologies of liposome preparation may be found in Liposome Technology (CFC Press New York 1984); Liposomes by Ostro (Marcel Dekker, 1987); Methods Biochem Anal. 33:337-462 (1988) and U.S. Pat. No. 5,283,185. Such methods include freeze-thaw extrusion and sonication. Both unilamellar liposomes (less than about 200 nm in average diameter) and multilamellar liposomes (greater than about 300 nm in average diameter) may be used as starting components to produce the complexes described herein.

By way of example, the lipids of choice (and any organic-soluble bioactive), can be dissolved in an organic solvent, mixed and dried onto the bottom of a glass tube under vacuum. The lipid film is rehydrated using an aqueous buffered solution containing the infected erythrocytes to be encapsulated by gentle swirling. The hydrated lipid vesicles can then be further processed by extrusion, submitted to a series of freeze-thawing cycles or dehydrated and then rehydrated to promote encapsulation of antigenic preparations. Liposomes can then be washed by centrifugation or loaded onto a size exclusion column to remove unentrapped bioactive components from the liposome formulation and stored at 4 °C. A basic method for liposome preparation is described in more detail in Thierry et al. (1992, Nuc. Acids Res. 20:5691-5698). Another method for preparation of a liposomal vaccine specific to parasitized red blood cells is provided in Stanisic et al. (2021, mBio 12:e02657-21).

In some examples, one or more cell-targeting ligands and/or lipid adjuvants can be at least partially encompassed in or otherwise associated with the lipid bilayer or lipid monolayer of the lipid vesicle (e.g., liposome, LNP). In such examples, an appropriate amount of the molecule can be included in the liposome preparation. The exact amount of cell-targeting ligand and/or adjuvant will be independently dependent on a range of properties, including but not limited to the affinity of the molecule to bind its target, the concentration of the target, the half-life of the molecule, etc.

In one example, the adjuvant is an aluminium-based adjuvant. For example, aluminium salts (alum) may be used as an adjuvant (e.g., aluminium phosphate, aluminium sulfate or aluminium hydroxide). In one example, the immunogenic compositions disclosed herein comprise aluminium phosphate, aluminium hydroxide or aluminium sulfate as an adjuvant. In certain examples, the adjuvant is or comprises aluminium hydroxide, such as an aluminium hydroxide wet gel suspension.

Examples of known suitable immune modulatory type adjuvants that can be used in mammals include, but are not limited to, saponin extracts from the bark of the Aquilla tree (QS21, Quil A), TLR4 agonists such as MPLA (Monophosphoryl Lipid A), 3D-MPLA (3-O-deacylated MPLA) or GLA-AQ, LT/CT mutants, cytokines such as the various interleukins (e.g., IL-2, IL-12) or GM-CSF, and the like.

Examples of known suitable immune modulatory type adjuvants with both delivery and immune modulatory features that can be used in humans include, but are not limited to, ISCOMS (see, e.g., Sjolander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

The immunogenic compositions disclosed herein may further comprise a surfactant. Suitable surfactants are known in the art and include, but are not limited to, polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one example, the surfactant is polysorbate 80. The final concentration of polysorbate 80 in the composition may be least 0.0001% to 10% (e.g., at least 0.0001%, 0.0005%, 0.001%, 0.005%, 0.01%, 0.05%, 0.1%, 0.5%, 1%, 5% or 10% or any range therein) or at least 0.001% to 1% or at least 0.01% to 1% polysorbate 80 weight to weight (w/w).

In one example, the final concentration of polysorbate 80 in the formulation is about 0.01%, 0.02%, 0.03%, 0.04%, 0.05%, 0.06%, 0.07%, 0.08%, 0.09% or 0.1% polysorbate 80 (w/w). In various examples, the final concentration of polysorbate 80 in the formulation is about 0.1%, 0.2%, 0.3%, 0.4%, 0.5%, 0.6%, 0.7%, 0.8%, 0.9% or 1% polysorbate 80 (w/w). In another example, the final concentration of the polysorbate 80 in the formulation is about 1% polysorbate 80 (w/w).

The immunogenic compositions disclosed herein may further comprise a buffer. The buffer may be any suitable buffer known in the art. For example, the buffer may be a TRIS, acetate, glutamate, lactate, maleate, tartrate, phosphate, citrate, carbonate, glycinate, histidine, glycine, succinate and triethanolamine buffer phosphate buffer. In one example, the buffer is a phosphate buffer. In another example, the buffer is a succinate buffer. In another example, the buffer is a histidine buffer. In another example, the buffer is a citrate buffer.

The buffer may be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The immunogenic compositions disclosed herein may further comprise one or more other antigens of other pathogens and/or parasites, particularly from bacteria, protozoa and/or viruses, as are known in the art.

It is envisaged that other components, such as immunologically or pharmaceutically acceptable carriers, diluents and/or excipients, may be included in the immunogenic composition described herein. Typically, these include solid or liquid fillers, diluents or encapsulating substances that maybe safely used in systemic administration. Depending upon the particular route of administration, carriers, diluents and/or excipients may be selected from a group including sugars, starches, cellulose and its derivatives, malt, gelatine, talc, calcium sulfate, vegetable oils, synthetic oils, polyols, alginic acid, isotonic saline, pyrogen free water, wetting or emulsifying agents, bulking agents, coatings, binders, fillers, disintegrants, lubricants and pH buffering agents (e.g. phosphate buffers) although without limitation thereto. The immunogenic composition may be administered to an animal in any one or more dosage forms that include tablets, dispersions, suspensions, injectable solutions, syrups, troches, capsules, suppositories, aerosols, transdermal patches and the like.

By “pharmaceutically acceptable carrier, diluent and/or excipient” or “immunologically acceptable carrier, diluent and/or excipient” is meant a solid or liquid filler, diluent or encapsulating substance that can be safely used in topical or systemic administration to an animal, preferably a mammal, including humans.

Formulations may be presented as discrete units such as capsules, sachets, functional foods/feeds or tablets each containing a pre-determined amount of one or more therapeutic agents of the present disclosure, as a powder or granules or as a solution or a suspension in an aqueous liquid, a non-aqueous liquid, an oil-in-water emulsion or a water-in-oil liquid emulsion. Such formulations may be prepared by any of the methods of pharmacy but all methods include the step of bringing into association one or more agents as described above with the carrier which constitutes one or more necessary ingredients. In general, the formulations are prepared by uniformly and intimately admixing the agents of the present disclosure with liquid carriers or finely divided solid carriers or both, and then, if necessary, shaping the product into the desired presentation.

Also disclosed herein is a container comprising the immunogenic compositions disclosed herein. Any suitable container known in the art may be used. For example, the container may be selected from the group consisting of a vial, a syringe, an ampoule, a flask, a fermentor, a bioreactor, a bag, a jar, an ampoule, a cartridge and a disposable pen. In one example, the container is a vial, an ampoule or a syringe.

The container may be made of glass, metals (e.g., steel, stainless steel, aluminium, etc.) and/or polymers (e.g., thermoplastics, elastomers, thermoplastic-elastomers). The container may be at least partially siliconized.

### Cell Targeting Ligands

In some examples, the immunogenic composition further comprises a cell targeting ligand. To this end, particles or lipid vesicles of the immunogenic composition (e.g., liposomes, LNPs) can be targeted to receptors on antigen presenting cells (APCs), for example, by placing ligands for cellular receptors of APCs on the surface of the particle (for example, mannosyl moieties or complement proteins such as C3d). For such examples, the immunogenic composition additionally comprises a cell targeting ligand at or on the surface of the particle or lipid vesicle. The cell-targeting ligand facilitates the delivery of the immunogenic composition to an immune cell, such as an APC. In some particular examples, the immune cell is an APC, such as a dendritic cell and/or a macrophage. In some other examples, the immune cell comprises a mannose receptor or a C-lectin type receptor on its cell surface.

Suitably, the cell-targeting ligand comprises a lipid anchor component, a linker component, and an oligosaccharide component.

In some examples, the oligosaccharide component comprises at least one mannosyl oligosaccharide or a functional fragment or derivative thereof. By way of example, the mannosyl oligosaccharide may comprise 1, 2, 3, 4, 5 or 6 mannose residues.

The lipid anchor component of the cell targeting ligand suitably binds, attaches to or otherwise integrates with at least one layer of the lipid bilayer or lipid monolayer of a lipid vesicle (e.g., liposome or LNP) of the immunogenic composition. The lipid anchor may comprise, consist, or consist essentially of, at least one lipid or fatty acid chain thereof. Suitably, the lipid is a C4-C20 lipid, or more particularly a C12-C18 lipid. By way of an illustrative example, the lipid may be a C16 lipid, such as palmitate. The lipid may be saturated or unsaturated, although suitably saturated.

The targeting moiety may further comprise a linker or spacer component. Suitably, the linker or spacer is located or positioned between the lipid anchor and the oligosaccharide component, such as a mannosyl oligosaccharide. The linker or spacer may comprise, consist, or consist essentially of one or more amino acids or peptides. Non-limiting examples of suitable amino acids include lysine and serine. In some examples, the linker or spacer may comprise polyethylene glycol. In certain examples, the spacer or linker may comprise one or more polyether compounds such as polyethylene glycol (PEG). The number of repeat units (O—CH₂—CH₂) may be 2-10 (e.g., 2, 3, 4, 5, 6, 7, 8, 9, 10 or any range therein). In some examples, the linker component comprises six repeat units (O—CH₂—CH₂). Suitably, the linker or spacer may comprise two or more linked units comprising the polyether compounds such as polyethylene glycol (PEG).

In one example, the targeting ligand is a mannosylated lipid core peptide (MLCP). Typically, the MLCP may be of the general form of the targeting ligands described above. Exemplary MLCP molecules designated F2-F5 are shown in Table 1 below.

TABLE 1

| Code | Oligosaccharide | Linker | Lipid |
|---|---|---|---|
| F2 | mannose | Lys-Lys-Ser-Ser | C16 |
| F3 | mannose | Lys-Lys-Ser-Ser | 2 x C16 |
| F4 | mannose | (PEG6)₂-Ser-Ser | C16 |
| F5 | (mannose)₄ | (Lys)₂-Lys-Ser-Ser | C16 |

In some particular examples in which the immunogenic composition is encapsulated in a lipid vesicle (e.g., a liposome, an LNP), the cell-targeting ligand is at least partially embedded in the lipid bilayer or lipid monolayer of the lipid vesicle (e.g., the lipid bilayer of a liposome, the lipid monolayer of an LNP). Suitably, the cell-targeting is at least partially embedded in the outer layer of the lipid bilayer or the lipid monolayer of the lipid vesicle.

In certain examples, the immunogenic composition comprises F3, DPPC, DDAB and cholesterol in a ratio of 10:5:2:1.

### Methods of Production

Related aspects of the present disclosure provides a method of producing an immunogenic composition, such as that described herein, including the steps of: (a) providing a plurality of erythrocytes that contain or are infected with an erythrocytic organism; and (b) contacting the erythrocytes with an antigen masking agent for a period of time and under conditions to facilitate the antigen masking agent conjugating, linking or otherwise associating with the erythrocytes.

Suitably, the present method further includes the initial step of producing, culturing or generating the erythrocytes infected with the erythrocytic organism. The erythrocytes may be obtained from blood of a donor subject infected with the erythrocytic organism (i.e., infected in vivo) prior to contacting with the antigen masking agent. Alternatively, to produce parasitized or infected erythrocytes or RBCs (i.e., pRBC) in vitro, non-infected red blood cells may be obtained from a suitable donor subject (e.g., a human donor) and then infected in vitro with the erythrocytic organisms so that the pRBC are thereafter treated or contacted with the antigen masking agent.

In an alternative form, the present disclosure provides a method of producing an immunogenic composition, such as that described herein, including the steps of: (a) providing a plurality of erythrocytes; (b) contacting the erythrocytes with an antigen masking agent for a period of time and under conditions to facilitate the antigen masking agent conjugating, linking or otherwise associating with the erythrocytes; and (c) contacting or infecting the erythrocytes with an erythrocytic organism. In relation to the present aspect, it is envisaged that step (c) may be performed before or after step (b).

According to particular examples, the present methods further include the further step of inactivating, attenuating and/or killing the erythrocytic organisms, such as by those methods described herein. Such a step may be performed before, after or simultaneously with the aforementioned step of contacting the erythrocytes with the antigen masking agent (i.e., step (b) above). In particular examples, the present step of inactivating, attenuating and/or killing the erythrocytic organisms is performed after step (b) above.

In certain examples, the present methods further include the further step of formulating the immunogenic composition such that the erythrocytes are contained in or otherwise associated with a particle, such as a lipid-based particle or a lipid-based vesicle, as hereinbefore described. In this regard, the erythrocytes may be contained in or otherwise associated with a liposome. In alternative examples, the erythrocytes are contained in or otherwise associated with an LNP. In some examples, the present step may include combining the erythrocytes with a vehicle agent, such as a cationic lipid or a lipid-based adjuvant. Such a method may further comprise combining the erythrocytes, or an associated particle or lipid vesicle (e.g., liposomes, LNPs), with a cell targeting ligand.

In some examples, the present methods include lysing the erythrocytes, such as by one or more (e.g., 1, 2, 3, 4, 5 etc) freeze-thaw cycles. In particular examples, the immunogenic composition has been frozen once, such as flash frozen with dry ice or liquid nitrogen, and then subsequently thawed prior to administration to a subject. Additionally, the present method may include lyophilising the immunogenic composition.

In view of the foregoing, the immunogenic composition may be conveniently prepared by a person skilled in the art using standard protocols, such as those hereinbefore provided. An exemplary method of preparing the immunogenic composition of the present disclosure is provided in Example 1.

In another broad form, the present disclosure provides an immunogenic composition produced by the method described herein.

### Administration

Suitable regimens for the administration of the immunogenic compositions disclosed herein are known in the art. The above compositions may be administered in a manner compatible with the dosage formulation, and in such amount as effective. The dose administered to the subject should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner, such as a medical or veterinary practitioner.

In some cases, as little as one dose of the immunogenic composition is needed, but under some circumstances, such as conditions of greater immune deficiency, a second, third or fourth dose may be given. Following an initial vaccination, subjects can receive one or several booster immunizations adequately spaced as required.

Accordingly, the methods described herein may comprise administering a priming dose of the immunogenic composition, wherein the immunogenic composition stimulates or otherwise enhances an immune response to an erythrocytic organism in a subject, and subsequently administering a later booster dose of the immunogenic composition as described herein.

For example, the booster dose may be administered at least 7, 14, 21 or 28 days, at least 1, 2, 3, 4, 5, or 6 months, or at least 1, 2, 3, 4, or 5 years after the priming dose. The priming and booster doses may be administered by the same or different routes. For example, the priming and booster doses may both be administered subcutaneously, intramuscularly, intravenously, or intraperitoneally. Alternatively, the priming dose may be administered locally (e.g., mucosally, such as intranasally) to induce mucosal antigen-specific immune cells, and the booster dose administered subcutaneously, intramuscularly, or intravenously to induce systemic antigen-specific immune cells. Suitably, the booster dose is administered intramuscularly.

In particular examples, a priming dose, such as one or more priming doses, of the immunogenic composition includes a first adjuvant and a booster dose, such as one or more booster doses, of the immunogenic composition includes a second adjuvant, which is different from the first adjuvant. For example, a priming dose of the immunogenic composition may comprise a lipid vesicle or a liposome and/or a lipid A glycolipid adjuvant, such as 3D(6-acyl)-PHAD®, whilst a booster dose of the immunogenic composition may comprise an aluminium-based adjuvant, such as alum.

Optimal amounts of components for a particular vaccine can be ascertained by standard studies involving observation of appropriate immune responses in subjects. For example, the dosage for human vaccination is determined by extrapolation from animal studies to human data. In another example, the dosage is determined empirically.

A typical dose of the immunogenic composition of the present disclosure for injection has a volume of 0.1 mL to 5 mL (e.g., 0.1, 0.2, 0.3, 0.4, 0.5, 0.6, 0.7, 0.8, 0.9, 1.0, 1.5, 2.0, 2.5, 3.0, 3.5, 4.0, 4.5, 5.0 mL or any range therein), more particularly 0.2 mL to 1 mL, even more particularly a volume of about 0.5 mL.

### Methods of Treatment

The immunogenic compositions disclosed herein or the peptides disclosed herein may be for use in a method of: (a) eliciting an immune response in a subject; and/or (b) preventing, treating or ameliorating an infection with an erythrocytic organism or a disease, disorder or condition associated therewith in a subject. Accordingly, the methods and compositions disclosed herein may have medical applications.

As used herein, “treating”, “treat” or “treatment” refers to a therapeutic intervention that at least partly ameliorates, eliminates or reduces a symptom or pathological sign of an erythrocytic organism-associated infection, disorder or condition, such as malaria, after it has begun to develop. It is also contemplated that the present methods may be used to treat or ameliorate latent or asymptomatic infections with the erythrocytic organism in a subject, as are known in the art. Treatment need not be absolute to be beneficial to the subject. The beneficial effect can be determined using any methods or standards known in the art.

As used herein, “preventing”, “prevent” or “prevention” refers to a course of action initiated prior to infection by, or exposure to, the erythrocytic organism and/or before the onset of a symptom or pathological sign of an erythrocytic organism-associated infection, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of an erythrocytic organism-associated infection, disorder or condition, or exhibits only early signs for the purpose of decreasing the risk of developing a symptom or pathological sign of an erythrocytic organism-associated infection, disease, or condition.

Thus, also disclosed herein is a method of preventing, treating or ameliorating an infection with an erythrocytic organism or a disease, disorder or condition associated therewith in a subject, said method including the step of administering to the subject a therapeutically effective amount of the immunogenic composition disclosed herein.

Also disclosed herein is the use of the immunogenic composition disclosed herein in the manufacture of a medicament for preventing, treating or ameliorating an infection with an erythrocytic organism or a disease, disorder or condition associated therewith in a subject.

Further described herein is a method of inducing or eliciting an immune response in a subject, the method comprising administering to the subject a therapeutically effective amount of the immunogenic composition described herein.

Also disclosed herein is the use of the immunogenic composition disclosed herein in the manufacture of a medicament for inducing or eliciting an immune response in a subject.

In a related form, the present disclosure provides a method immunizing a subject against an infection, disease or condition associated with an erythrocytic organism, comprising administering to the subject a therapeutically effective amount of the immunogenic composition disclosed herein.

Also disclosed herein is the use of the immunogenic composition disclosed herein in the manufacture of a medicament for immunizing a subject against an infection, disease or condition associated with an erythrocytic organism.

The term “therapeutically effective amount” describes a quantity of a specified agent, such as immunogenic composition described herein, sufficient to achieve a desired effect in a subject being treated with that agent. For example, this can be the amount of the immunogenic composition, necessary to elicit an immune response in the subject, immunize the subject against the erythrocytic organism and/or prevent, treat or ameliorate an infection, disease or condition associated with the erythrocytic organism. Suitably, a “therapeutically effective amount” is sufficient to prevent, reduce or eliminate a symptom of an infection with the erythrocytic organism. More particularly, a “therapeutically effective amount” may be an amount sufficient to achieve a desired biological effect, for example an amount that is effective to decrease or prevent disease or infection progression.

Ideally, a therapeutically effective amount of an agent is an amount sufficient to induce the desired result without causing a substantial cytotoxic effect in the subject. The effective amount of an agent useful for reducing, alleviating and/or preventing an infection of the erythrocytic organism will be dependent on the subject being treated, the type and severity of any associated disease, disorder and/or condition (e.g., disease progression), and the manner of administration of the therapeutic composition.

In particular examples, the present methods exclude the step of administering an adjuvant to the subject. This is particularly so for those examples in which the immunogenic composition comprises intact erythrocytes infected with erythrocytic organisms that have been attenuated, such as chemically attenuated.

So that the present disclosure may be described in detail and put into practical effect, reference is made to the following non-limiting Examples.

### EXAMPLES

### Example 1—Protocol for Producing Killed Whole Parasite Malaria Vaccine with Antigen-Masked RBCs

mPEG=Methoxypolyethylene glycol activated with cyanuric chloride
1. Prepare PEG buffer. This is isotonic alkaline phosphate buffered saline—50 mM K2HPO4/105 mM NaCl, pH 8.0.
2. Prepare a 6 mg/ml solution of mPEG by dissolving 6 mg of mPEG in 1 ml of PEG buffer.
3. Determine the parasitemia of the parasitised red blood cells to be used by preparing and reading a blood film.
4. Add 120 ul of parasitised red blood cells into 20 ml of PEG buffer in a 50 ml tube. Perform reactions in multiples of 120 ul. Note: For P. falciparum, these parasitised red blood cells are generally purified using a VARIOMACs magnet with purity >90%. For rodent malaria parasites, the parasitised red blood cells are generally not purified.
5. Centrifuge at 1500 rpm, brake 0, for 5 minutes at room temperature.
6. Carefully remove supernatant (taking care not to disturb the pellet). Discard supernatant.
7. Repeat wash steps 5-6 a further two times (i.e., 3 washes in total).
8. After removing the supernatant from the final wash, resuspend up to 1 ml with PEG buffer Perform a cell count for each reaction tube using a hemocytometer and trypan blue exclusion. Calculate the number of parasitised red blood cells using the cell count and the parasitemia from the blood smear.
9. Centrifuge at 1500 rpm, brake 0, for 5 minutes at room temperature.
10. Remove supernatant (taking care not to disturb the pellet). Discard supernatant.
11. Add the 120 ul of the washed parasitised red blood cells to 880 ul of a 6 mg/ml mPEG solution. (Hematocrit of solution is approximately 12%).
12. Gently mix with a transfer pipette (10 times) to ensure homogenous modification of the red blood cells.
13. Incubate for 1 hour at room temperature.
14. Top tube up to 20 ml with commercial D-PBS.
15. Centrifuge at 4500 rpm, brake 0, for 5 minutes at room temperature.
16. Carefully remove supernatant (taking care not to disturb the pellet). Discard supernatant.
17. Repeat wash steps 15-16 a further 2 times (i.e., 3 washes in total) 18. After removing the supernatant from the final wash, combine all pellets in a single tube.
19. Top up to 20 ml with commercial D-PBS.
20. Centrifuge at 4500 rpm, brake 0, for 5 minutes at room temperature.
21. Remove supernatant and perform 6 cycles of freeze thawing of the pellet.
22. Record the total number of parasites (adding together the number of parasitised red blood cells for the separate reaction tubes if needed) and the date on the final tube. After the final cycle, place at −80 °C for storage until required.

### Example 2—Efficacy and Immunogenicity of Killed Whole Parasite Malaria Vaccine with Antigen-Masked RBCs

### Materials & Methods

### Vaccine Production

The killed whole parasite malaria vaccine with antigen-masked RBCs was produced as per the above protocol. The vaccine was further adjuvanted and formulated into liposomes, as per the protocol described in Stanisic et al. (2021, mBio 12:e02657-21).

Flow Cytometry-Based Method to Confirm Successful mPEG Treatment
1. Transfer 1×10⁶ red blood cells into 200 μl MACS buffer (2 mM EDTA, 0.5% bovine serum albumin in PBS) in an eppendorff tube. Prepare three tubes for each experiment: unstained, mPEG treated cells for staining, untreated cells for staining.
2. Prepare fluorophore conjugated antibodies as follows (per sample)
   TER119: 5 μl TER119 to 95 μl MACS buffer (mouse red blood cells)
   RhD/CE: Sul RhD to 95 μl MACS buffer (human red blood cells
   100 μl MACs buffer to unstained samples
3. Centrifuge red blood cells at 14,800 rpm for 10 minutes.
4. Discard supernatant and resuspend cells in the appropriate fluorophore-conjugated antibodies (for staining) or in MACS buffer by pipetting gently 10 times.
5. Incubate in the dark for 30 minutes.
6. Add 900 μl of MACS buffer to each tube and wash at max speed (14,800 rpm) for 10 minutes.
7. Discard supernatant. Repeat this wash step a further 2 times.
8. Add 300 μl MACS buffer to each sample and transfer to FACS tube for analysis on the flow cytometer.

### Serum Binding Assay (Flow Cytometry-Based Method) to Measure Anti-Red Blood Cell Antibodies in Mice

1. Wash 1 ml of human normal red blood cells (nRBCs) with 20 ml tissue culture medium (RPMI-1640+L-Glutamine+Gentamycin).
2. Centrifuge 400 g/5 mins/room temperature (RT)/acceleration: 6, deceleration: 0
3. Remove supernatant.
4. Repeat steps 1-3 two times (total 3 wash steps).
5. Prepare nRBC suspension at 0.2% haematocrit (8.241 washed nRBC+4.1 ml tissue culture medium.
6. Mix 10 μl neat serum (derived from mice) to 90 μl of the nRBC suspension prepared in step 5, in a 1.5 ml eppendorff tube.
7. Mix gently with a pipette.
8. Incubate 30 mins at room temperature
9. Add 100 μl of a 1% FCS/PBS to each tube and transfer contents to a well in a 96 well U-bottom tissue culture plate.
10. Centrifuge 400 g/5 mins/RT/acceleration: 6, deceleration: 0 11. Remove supernatant.
12. Repeat wash step.
13. Add 50 μl of goat anti-mouse IgG ALexaFluor-488 (1:1000 dilution in MACs buffer) to all samples, except for an unstained control.
14. Incubate for 30 mins at room temperature in the dark.
15. Wash samples 3 times: 200 μl MACS buffer, spin 400 g/5 mins/RT/acceleration: 6, deceleration: 0
16. Resuspend in 150 μl of MACs buffer and transfer samples to FACS tubes containing 150 μl of MACs buffer.
17. Acquire sample on flow cytometer.
18. Acquire at least 50,000 events in the red blood cell gate.

The remaining materials and methods were essentially performed as outlined in Stanisic et al. 2021, mBio, which is incorporated by reference herein in its entirety.

### Results

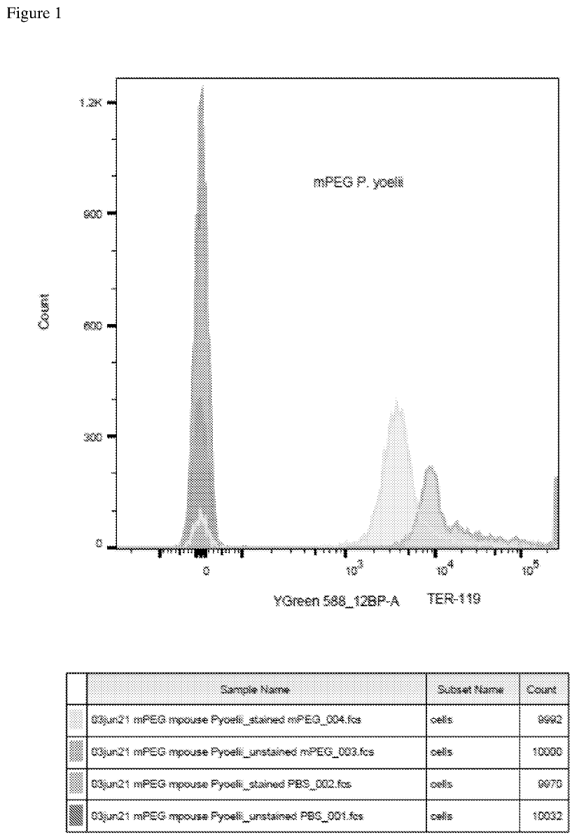

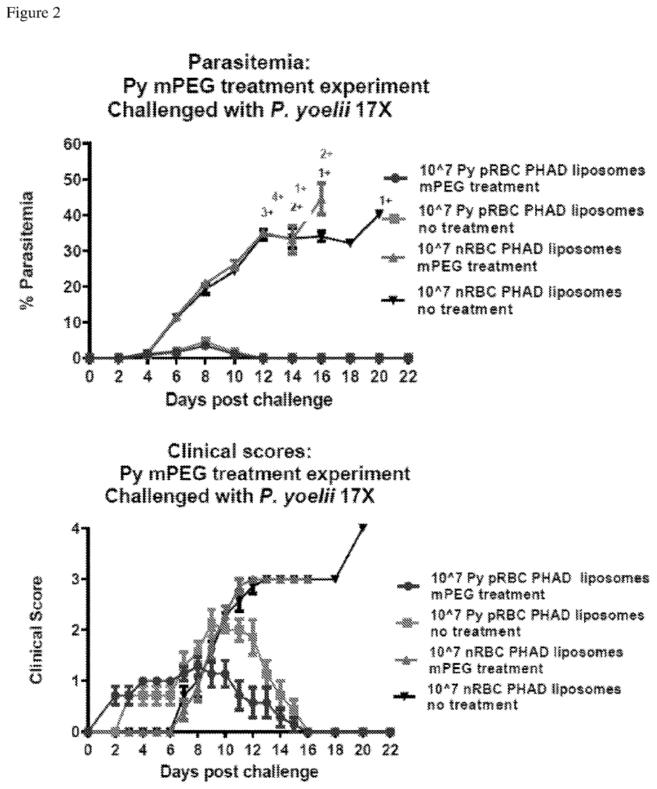

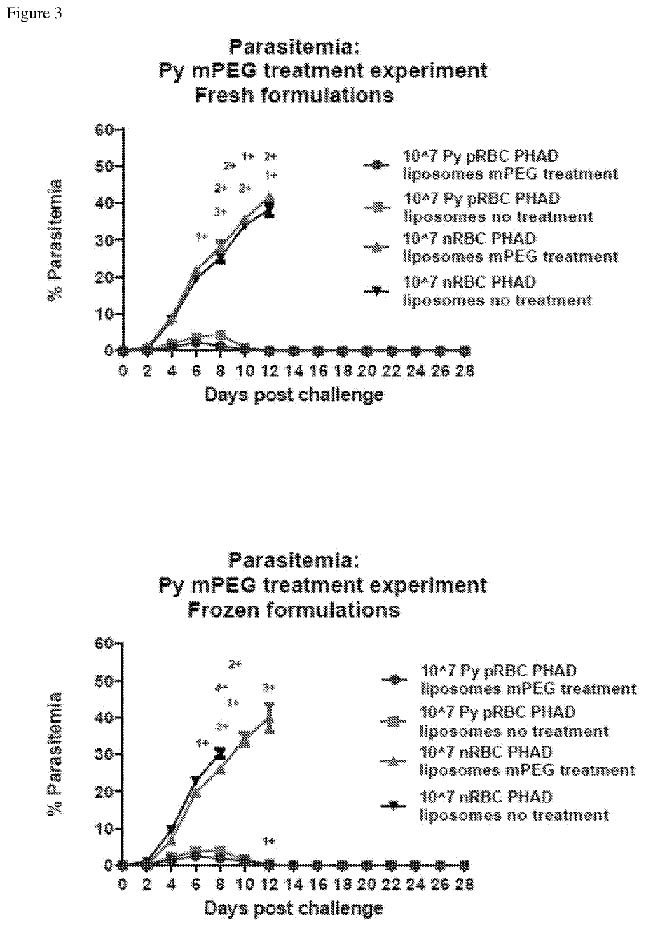

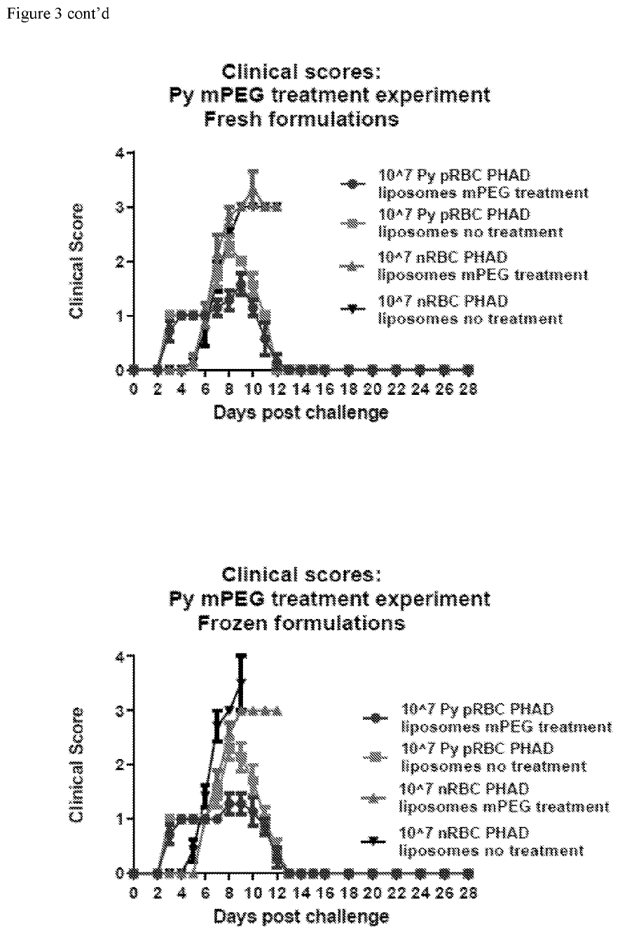

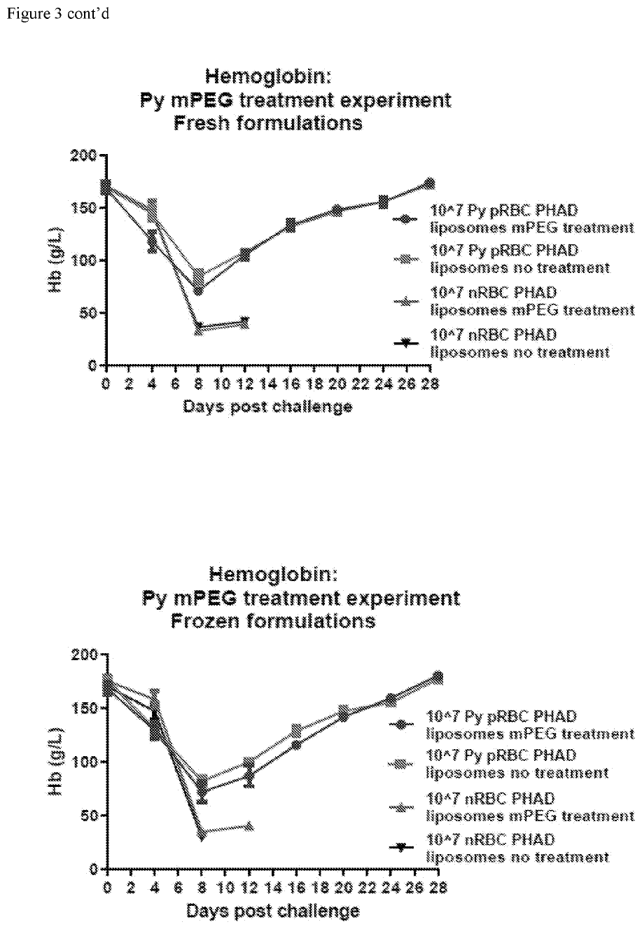

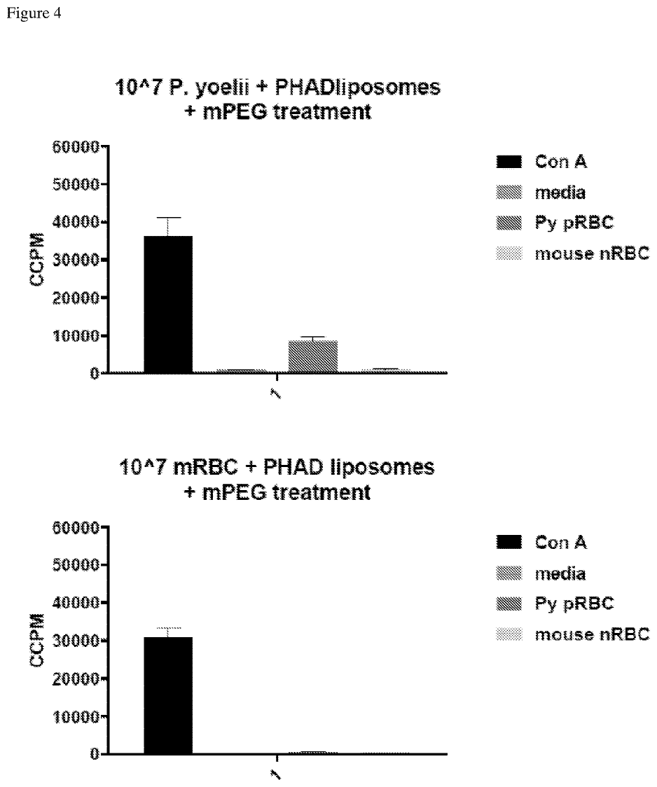

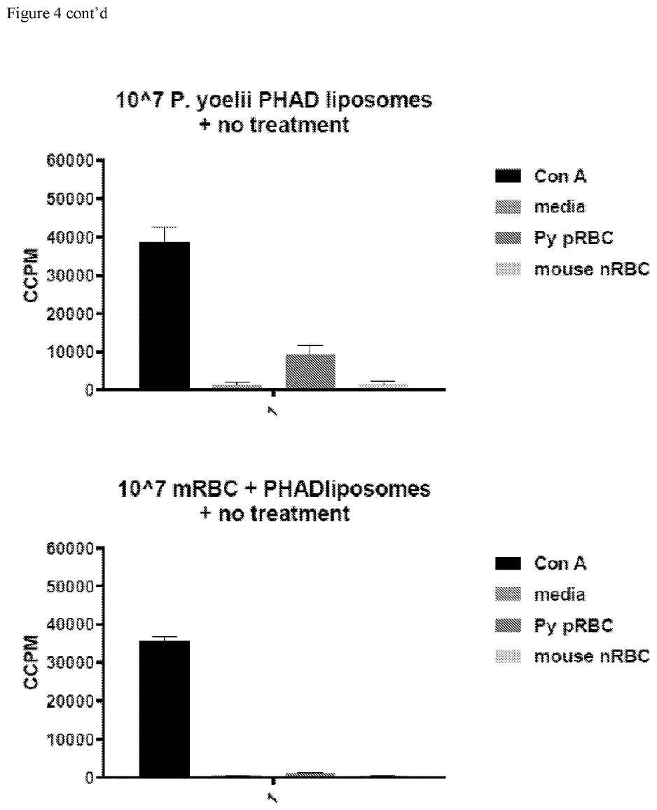

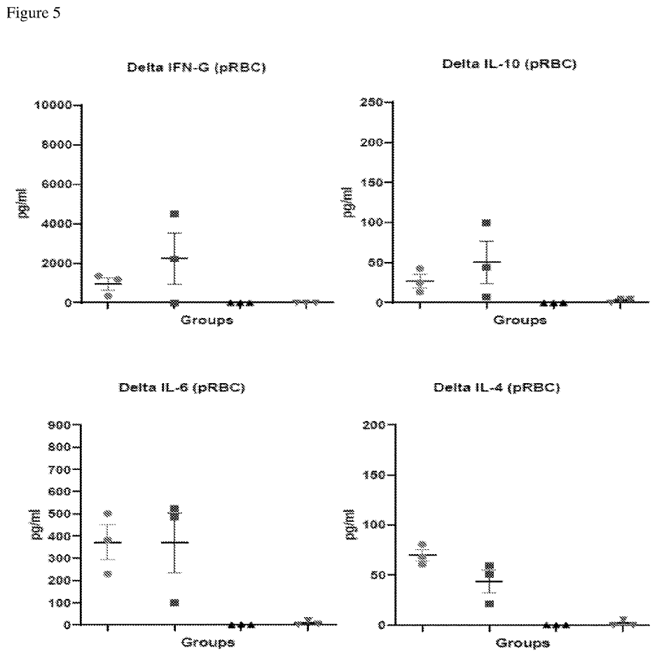

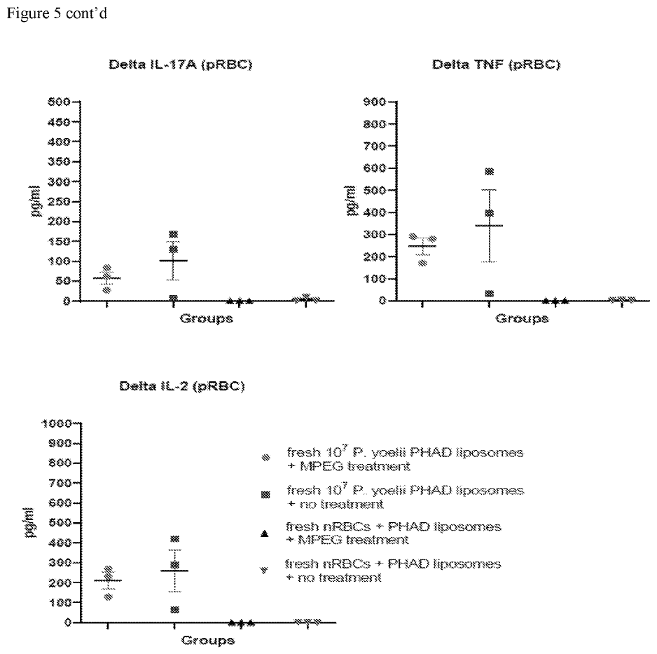

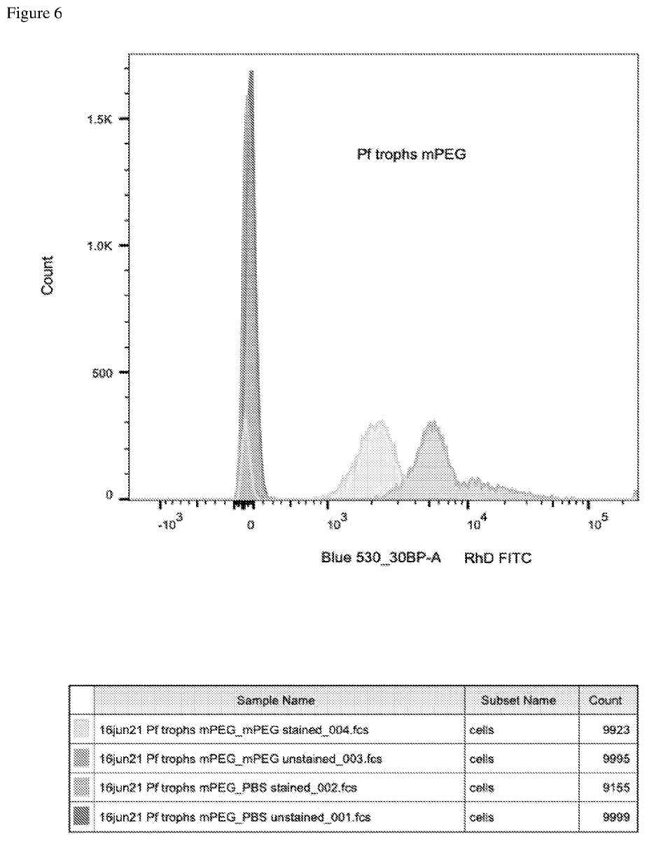

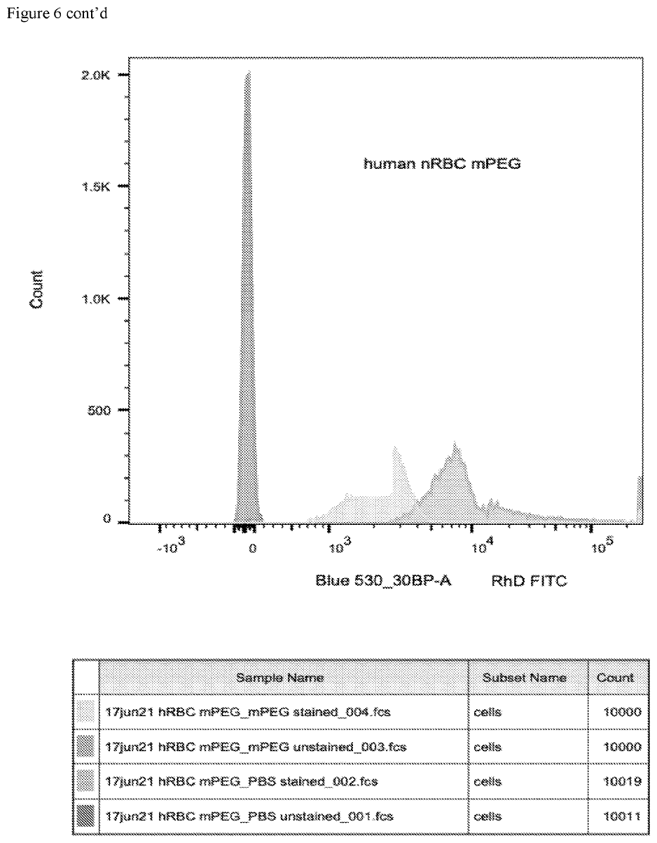

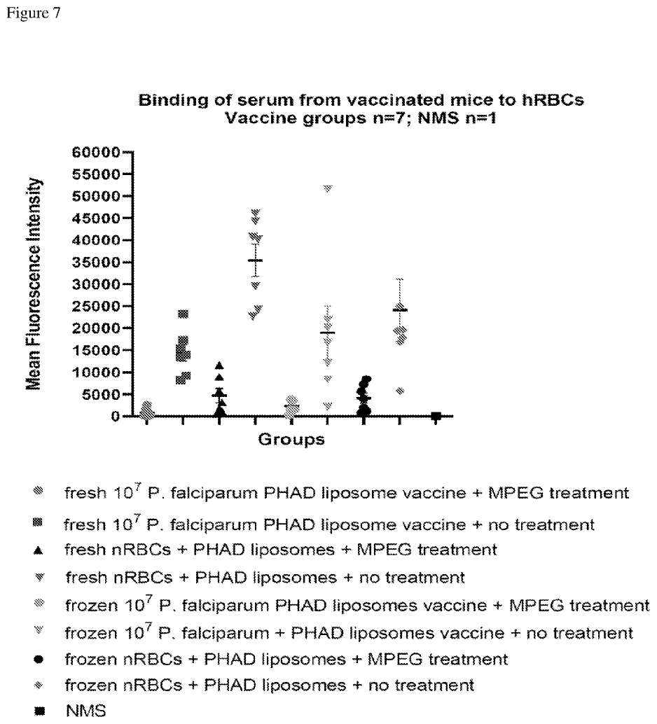

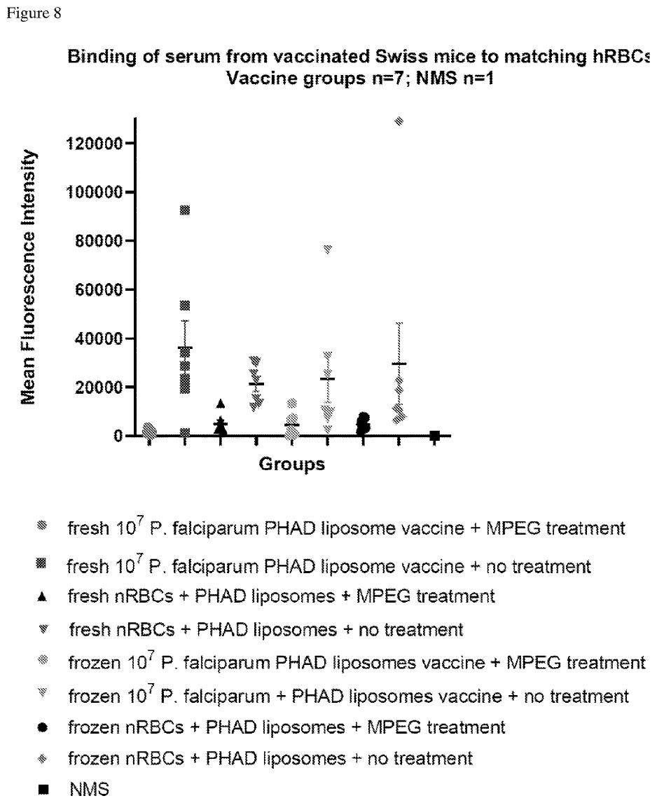

Treatment with mPEG-cyanuric chloride reduces the detectability by flow cytometry of red blood cell antigens (TER-119; Rhd) on the surface of mouse red blood cells (FIG. 1) and purified P. falciparum infected human red blood cells (FIG. 6).
In a rodent model of malaria, mPEG-cyanuric chloride treatment of P. yoelii parasitised red blood cells prior to formulating a whole blood-stage parasite malaria liposomal vaccine therefrom does not impact on the efficacy of fresh or cryopreserved vaccine formulations. Here, efficacy is evaluated based on control of the parasite following challenge in the rodent model (i.e., parasitemia levels, clinical scores and haemoglobin levels indicative of parasitic haemolysis) (FIGS. 2 and 3).
Surprisingly, the mPEG-based whole parasite vaccine also results in a significant reduction in the clinical scores of vaccinated mice following challenge (versus the clinical scores of mice vaccinated with a control whole parasite vaccine lacking mPEG) (FIGS. 2 and 3). Without being bound by any theory, it is hypothesised that the mPEG, in addition to masking RBC antigens, is also masking malarial antigens in the RBC membrane that are involved, for example, in immune evasion, but are not necessarily important in immune protection from the malarial parasites. As such, the presence of mPEG could also be preventing or blocking any negative immune or inflammatory responses by the host in response to these RBC-based antigens of the parasite that may contribute to the pathological response seen in malarial disease.
In a rodent model of malaria, mPEG-cyanuric chloride treatment of P. yoelii parasitised red blood cells prior to formulating a whole blood-stage parasite malaria liposomal vaccine does not impact on parasite-specific splenocyte proliferative responses or cytokine responses (FIGS. 4 and 5).
mPEG-cyanuric chloride treatment of P. falciparum pRBCs in a whole blood-stage parasite malaria vaccine significantly reduces production of antibodies that recognise the surface of normal human red blood cells in genetically inbred and outbred mice (FIGS. 7 and 8).

In conclusion, the present example has provided valuable data regarding vaccine characteristics, immunogenicity and efficacy for a malaria vaccine that includes RBCs of reduced immunogenicity.

### REFERENCES

1. Stanisic D I, Ho M-F, Nevagi R, Cooper E, Walton M, Islam M D, Hussein W M, Skwarczynski M, Toth I, and Good M F 2021 Development and evaluation of a cryopreserved whole-parasite vaccine in a rodent model of blood-stage malaria. mBio 12: e0265721

### Example 3—a Six Week Repeat Dose Toxicity Study in Sprague Dawley Rats with a Malaria Vaccine Administered by Intramuscular Injection

The objective of the present Example study was to assess the toxicity of an adjuvanted malaria vaccine of the present disclosure when administered every two weeks by the intramuscular route to Sprague Dawley rats for a six week study period. The reversibility of any treatment-related changes was also assessed after a treatment-free two-week recovery period.

### Materials & Methods

### Test Item and Control Formulation

The formulations were prepared on the day of dosing by mixing the antigen with CAF01/Alhydrogel adjuvant as outlined in Table 1 below.

TABLE 1
Dose Formulation Procedures

| Group | Treatment | Formulation Procedures |
|---|---|---|
| 1 | CAF01 + 10 mM tris Buffer | CAF01 diluted 1:1 (v/v) with Tris Buffer |
| | Alhydrogel + PBS | PBS adsorbed to Alhydrogel sent by the Sponsor |
| 2 | Malaria antigen + CAF01 | Malaria antigen diluted 1:1 (v/v) with CAF01 |
| | Malaria antigen + Alhydrogel | mPEG treated P. falciparum lysate adsorbed to |
| | | Alhydrogel were sent by the Sponsor |

The formulations described above were prepared in a laminar flow hood using sterile polypropylene tubes. The formulations were subjected to vortex mixing. The PBS adsorbed to Alhydrogel formulation and mPEG treated P. falciparum lysate adsorbed to Alhydrogel formulation were mixed by several inversions.

The adjuvant and malaria vaccine dose rates, based on administration of 2×100 μL per rat, and dosing days are summarised in Table 2.

TABLE 2
Dose Rates and Dosing Days

| | | | Dose Rates |
|---|---|---|---|
| | | | (2 x 100 μL/ |
| Group | Treatment | Dosing Days | dose per rat) |
| 1 | CAF01 + 10 mM tris Buffer | 1, 15, 29 | 200 μL |
| | Alhydrogel + PBS | 43 | 200 μL |
| 2 | Malaria antigen + CAF01 | 1, 15, 29 | 200 μL |
| | Malaria antigen + Alhydrogel | 43 | 200 μL |

The adjuvant and vaccine test item formulations were maintained at room temperature on each day of use and used within two hours of preparation on the same day. Any unused formulation was discarded after use.

### Animals

| | |
|---|---|
| Species | Rat |
| Strain | Sprague Dawley |
| Source | Ozgene (Head Office) |
| | Post Box 1128 |
| | Bentley DC |
| | WA 6983, Australia |
| Number of Animals | 60 Rats (30 males + 30 females) |
| Sex | Males and females (females were nulliparous and non-pregnant) |
| Weight at Start of Dosing | Males: 233.32-280.00 g |
| | Females: 184.51-235.77 g |
| Age at Start of Dosing | 6-8 weeks |
| Identification | Animals were identified by unique animal numbers which were |
| | associated with a Thermal microchip implanted subcutaneously into |
| | each animal at the time of receipt. Destron bio thermal microchip was |
| | not used as specified in the Study Plan rather an equivalent thermal |
| | microchip was used. A scanner was used to read the microchip prior to |
| | all procedures. |
| | Tails were marked with permanent pen for identification within cages, |
| | but these marks were not used as the primary identifier for any animal. |
| Randomisation | Animals were randomly assigned to treatment groups, with |
| | stratification according to body weight, on the day of arrival using |
| | Provantis. |
| Acclimation | Animals were subjected to a minimum seven-day acclimation period |
| | prior to dose administration. This acclimation period included a health |
| | examination and only animals without visible signs of illness were |
| | used for the study. |

### Study Design

The design of this six-week repeated dose toxicity study in Sprague Dawley rats was adapted from the recommendations of the World Health Organisation ‘Guidelines on the nonclinical evaluation of vaccine adjuvants and adjuvanted vaccines’ 2014 (1) and also the OECD Guideline for Testing of Chemicals No. 407 ‘Repeated Dose 28-Day Oral Toxicity Study in Rodents’ 2008 (3). The study procedures were conducted as outlined below and according to related test facility Standard Operating Procedures.

The study design and animal number are summarised in Tables 3 and 4 below.

TABLE 3
Study Design

| | | | | | Termi- |
|---|---|---|---|---|---|
| | | | Sprague | Treatment | nation |
| Group | Treatment | Allocation | Dawley Rats | Days | Day |
| 1 | Adjuvant | Main | 10 M + 10 F | 1, 15, 29, | 44 |
| | only | | | 43 | |
| | | Recovery | 5 M + 5 F | 1, 15, 29, | 58 |
| | | | | 43 | |
| 2 | Adjuvenated | Main | 10 M + 10 F | 1, 15, 29, | 44 |
| | Vaccine | | | 43 | |
| | | Recovery | 5 M + 5 F | 1, 15, 29, | 58 |
| | | | | 43 | |

TABLE 4
Animal Number

Animal Numbers

| Group | Treatment | Males | Females |
|---|---|---|---|
| 1 | Adjuvant | 1-10 (Main) | 31-40 (Main) |
| | only | 11-15 (Recovery) | 41-45 (Recovery) |
| 2 | Adjuvenated | 16-25 (Main) | 46-55 (Main) |
| | Vaccine | 26-30 (Recovery) | 56-60 (Recovery) |

Briefly, on completion of the acclimation period, the study commenced with administration of the treatments on Study Day 1, with additional treatments administered on Study Days 15, 29 and 43 for Groups 1 and 2.

On Study Days 1, 15 and 29 the rats in Group 1 were administered with (CAF01+10 mM Tris Buffer) and on Study Day 43 with (Alhydrogel+PBS). The rats in Group 2 were administered malarial antigen mixed with CAF01 on Study Days 1, 15 and 29 and antigen adsorbed with Alhydrogel on Study Day 43.

The commencement of the study was staggered with the males treated approximately one week prior to the females. All animals were observed for signs of toxicity throughout the in-life study period. The main study animals were subjected to terminal examinations on Study Day 44 for Group 1 and 2 after administration of the final dose and the recovery allocation animals were allowed a treatment free period of two weeks prior to terminal examinations on Study Day 58. The study procedures are detailed further below.

### Treatments

The adjuvant only and vaccine treatments were administered as a bolus dose by the intramuscular route in a total volume of 200 L/rat, divided between two equal injections (100 μL) at sites in the left and right upper thigh muscle on Study Days 1, 15, 29 and 43 for Groups 1 and 2. The doses were administered in the presence of light gaseous isoflurane anaesthesia. The fur at the injection site area (approximately 1 cm×1 cm) remained clipped throughout the study to aid in identification of injection site.

### Observations

Morbidity and mortality observations were recorded at least once daily during the acclimation period and termination days (Study Days 44 and 58), and twice daily from Study Day 1 through Study Day 43 during treatment and recovery periods.

Clinical observations were conducted at least once during the acclimation period and at least once daily during the treatment and recovery periods up to and including the day prior to termination. On treatment days the observations were completed at 30-60 min after treatment.

The clinical observations included examination of animals for changes in skin and fur, eyes and mucous membranes, respiratory and circulatory function, gait and posture, behaviour, tremors or convulsions and any other abnormal findings. This also included daily examination of the injection site for reactions. Any other injection site lesions such as eschar, necrosis and ulceration were also noted.

### Body Weights

Body weight for all animals were recorded during the acclimation phase, weekly during the treatment and recovery periods and on the day of necropsy.

### Food Consumption

Food consumption was recorded weekly.

### Body Temperature

Body temperature was recorded at least once during the acclimation period and at 3 to 8 h after each dose. If an increase in body temperature greater than 1.5 °C above the acclimation readings was detected post-dose the body temperature measurements were continued for every 24 h until return to within ±1.5 °C of the baseline measurement. Body temperature measurements were collected using subcutaneously implanted Bio Thermal microchips.

### Clinical Pathology—Haematology, Biochemistry and Coagulation

The rats were fasted with access to food withdrawn, but water available ad libitum, on the evening prior to termination (fasting commenced no earlier than 4 μm on each occasion). After administration of an overdose of pentobarbitone (Lethabarb; via intraperitoneal injection) to anaesthetise the animal, terminal blood samples were collected by puncture of the inferior vena cava. The resulting blood sample was immediately divided into four aliquots for analyses as follows:
Haematology: ~1.0 mL of blood was transferred into a K3-EDTA tube and stored at 2-8 °C.
Coagulation: ~1.0 mL of blood was transferred into a 1.0 mL sodium citrate tube, processed to plasma and the resulting sample transferred to a receiving tube and stored at 2-8 °C;
Biochemistry: ~2.0 mL of blood was transferred into a serum separator tube and subsequently processed to serum and the resulting sample transferred to a receiving tube and stored at 2-8 °C.
Immune response: ~1.0 mL of blood was transferred into a serum separator tube and subsequently processed to serum and the resulting sample transferred to two receiving tubes (each containing approximately 200 μL) and stored at −80 °C.

### Haematology

The following haematology parameters were determined at the Test Facility in K3-EDTA treated whole blood samples using an Idexx ProCyte Haematology Analyser within 24 h of collection:
Haemoglobin
Red blood cell count
Haematocrit
Mean Cell Volume
Mean Cell Haemoglobin Concentration
Mean Cell Haemoglobin
Reticulocyte counts (% and absolute)
White blood cell count
Differential White Cell count with cell morphology:
   Neutrophil
   Lymphocyte
   Monocyte
   Eosinophil
   Basophil
Platelet count

Blood smears were prepared and examined microscopically as required. Slide review was only performed on samples that meet flagging criteria in order to confirm accurate haematology analyser results. The whole blood samples were discarded after sample analysis. The study pathologist was provided with the haematology data for correlation with the microscopic findings.

### Biochemistry

The following biochemistry parameters were determined (or calculated) in serum samples using an Idexx Catalyst Clinical Chemistry Analyser within 24 h of collection:
Sodium
Potassium
Chloride
Calcium
Inorganic phosphorus
Glucose
Urea
Creatinine
Creatinine phosphokinase
Total bilirubin
Total cholesterol
Triglycerides
Alkaline phosphatase
Aspartate aminotransferase
Alanine aminotransferase
Total proteins
Albumin/globulin ratio
Albumin
Globulin

### Coagulation

The following coagulation parameters were determined in citrated plasma samples using a Stago STA Satellite Max Coagulation Analyser within 24 h of collection:
Prothrombin time (PT)
Activated Partial Thromboplastin Time (APTT)

### Necropsy

On the scheduled day of termination each rat was weighed and anaesthetised by administration of an overdose of pentobarbitone (via intraperitoneal injection) and a terminal blood sample collected for haematology (whole blood), coagulation (plasma), biochemistry (serum) and immunogenicity (serum) analyses as detailed above. Death resulted from exsanguination.

The necropsy procedures for male and female animals in each group was performed on n=10 animals per group per sex on Study Days 44 and n=5 animals per group per sex on Study Day 58. The blood samples collected on the day of termination were processed for clinical pathology analyses.

The necropsy procedures were supervised by a veterinarian and included a detailed examination of the external appearance of the animal and internal organs. All macroscopic observations were recorded. The necropsy included a record of the weights of critical organs and collection and preservation of the tissues and organs. Paired organs were weighed together. Organ weights were reported as absolute values and as organ to terminal body weight ratios.

Tissues were collected in 10% neutral buffered formalin with the following exceptions: the testes were collected in modified Davidson's fixative and eyes plus attached optic nerves in Davidson's fixative. At approximately 48 hours post-collection the testes and eyes plus attached optic nerves were briefly washed with tap water and then transferred to 10% neutral buffered formalin for storage prior to trimming and processing. Bone marrow smears were prepared but not analysed.

### Histopathology

Tissues from n=10 males and n=10 females from Groups 1 and 2 euthanised on Study Day 44 and tissues from n=5 males and n=5 females euthanised on Study Day 58 (n=60 total) and any gross lesions were subjected to microscopic examination. These tissues were trimmed, embedded in paraffin, sectioned, mounted on glass slides and stained with haematoxylin and eosin per test site standard operating procedures (SOPs).

### Results

Treatment with the adjuvanted vaccine was well tolerated post repeated administration on Study Days 1, 15, 29 and 43. Although, clinical findings of mild chromodacryorrhea in the nose and eyes were observed in the adjuvanted vaccine group, the incidence and severity of these findings were comparable to rats in the adjuvant only group and therefore these findings are considered unrelated to treatment.

There were no treatment related changes observed in the body weights of rats in the adjuvanted vaccine group. There were no effects of treatment on either sex on food consumption relative to the adjuvant only treated rats. Administration of adjuvanted vaccine were not associated with any changes in body temperature relative to adjuvant only treated rats on Study Days 1, 15, 29 or 43.

Increases in white blood cell (WBC) counts were noted in rats of adjuvanted vaccine Group 2 on Study Day 44; which microscopically correlated with mononuclear or mixed inflammation in the muscle and/or mononuclear or mixed inflammation in the subcutis.

Statistically significant decreases in albumin to globulin (A:G) ratio were observed in both sexes of adjuvanted vaccine Group 2 on Study Day 44 and 58 compared to adjuvant only Group 1. This decrease in (A:G) ratio is considered a result of inflammatory response which microscopically correlated with mixed inflammation observed in muscle or subcutis.

Adjuvanted vaccine Group 2 males showed a statistically significant lower ALT mean, however a similar difference was not noted in females. Therefore, the reported disturbances in ALT for males, was considered of a doubtful relationship to treatment and not of toxicological importance.

Statistically significant decreases in Activated Partial Thromboplastin Time (APTT) were observed in males of adjuvanted vaccine relative to adjuvant only group on Study Day 44.

There was increased incidence of macroscopic observations at the injection sites of males administered adjuvanted vaccine (Group 2) relative to adjuvant only (Group 1) males. These macroscopic observations typically included pale/red discoloration of the subcutaneous tissue and/or musculature at the injection sites, sometimes accompanied by a firm or nodular area. Microscopically, these observations correlated with mononuclear or mixed inflammation in the muscle and/or mononuclear or mixed inflammation in the subcutis. Following the 2-week recovery period, there was no notable resolution of injection site macroscopic observations for either group. In females, macroscopic observations at the injection sites occurred with a similar incidence (100%) in groups 1 and 2 at the end of the main study and recovery period.

Microscopic observations at the injection sites that were exacerbated by the adjuvanted vaccine were mononuclear cell inflammation in the muscle and mixed or mononuclear cell inflammation in the subcutis. At the end of the two-week recovery period, mixed inflammation in the subcutis had completely resolved in the left and right injection sites of all animals in all groups. Mononuclear cell inflammation in the muscle and subcutis remained slightly exacerbated at the right injection sites of animals administered with adjuvanted vaccine compared to those that received adjuvant only. However, the incidence of microscopic findings in the left injection sites at the recovery period were similar between groups 1 and 2, indicating that the adjuvanted vaccine exacerbated microscopic changes to a lesser degree by the end of the recovery period. A recovery process was underway at the injection site.

In conclusion, the treatments with mPEG treated P. falciparum 7G8 lysate with CAF01 on Study Day 1, 15, and 29 or mPEG treated P. falciparum lysate adsorbed to Alhydrogel on Study Day 43 by the intramuscular route in the conditions of this six-week study were well tolerated.

1. An immunogenic composition comprising:
a plurality of erythrocytes;
a plurality of erythrocytic organisms; and
an antigen masking agent conjugated, linked or otherwise associated with the erythrocytes.

2. The immunogenic composition of claim 1, wherein the antigen masking agent is or comprises a polyethylene glycol or a derivative thereof.

3. The immunogenic composition of claim 1, wherein the antigen masking agent is or comprises methoxypolyethylene glycol (mPEG).

4. The immunogenic composition of claim 1, wherein the immunogenic composition further comprises an adjuvant.

5. The immunogenic composition of claim 4, wherein the adjuvant is or comprises a lipid A glycolipid adjuvant or a derivative thereof.

6. The immunogenic composition of claim 5, wherein the lipid A glycolipid adjuvant is a synthetic monophosphoryl 3-deacyl lipid A adjuvant or a native monophosphoryl lipid A adjuvant.

7. The immunogenic composition of claim 1, wherein the erythrocytes have been previously infected with the erythrocytic organism.

8. The immunogenic composition of claim 1, wherein the erythrocytic organisms have been attenuated, inactivated and/or killed.

9. The immunogenic composition of claim 1, wherein the erythrocytic organisms have been chemically attenuated or inactivated.

10. The immunogenic composition of claim 9, wherein the erythrocytic organisms have been chemically attenuated with a DNA binding agent.

11. The immunogenic composition of claim 10, wherein the DNA binding agent is selected from centanamycin, tafuramycin A and any combination thereof.

12. The immunogenic composition of claim 1, wherein the erythrocytic organisms have been killed.

13. The immunogenic composition of claim 1, wherein the erythrocytes are intact.

14. The immunogenic composition of claim 1, wherein the erythrocytes are lysed.

15. (canceled)

16. The immunogenic composition of claim 1, wherein the erythrocytic organism is selected from the group consisting of a Plasmodium sp., a Babesia sp., an Anaplasma sp., an Ehrlichia sp., a Trypanosoma sp., a Theileria sp., a Hepatozoon sp., a Mycoplasma sp., a Bartonella sp. and any combination thereof.

17. The immunogenic composition of claim 16, wherein the erythrocytic organisms are or comprise a Plasmodium sp.

18. (canceled)

19. The immunogenic composition of claim 1, wherein the erythrocytes are contained in or otherwise associated with a liposome.

20-22. (canceled)

23. A method of preventing, treating or ameliorating an infection with an erythrocytic organism or a disease, disorder or condition associated therewith in a subject, said method including the step of administering to the subject a therapeutically effective amount of an immunogenic composition comprising:
a plurality of erythrocytes;
a plurality of erythrocytic organisms; and
an antigen masking agent conjugated, linked or otherwise associated with the erythrocytes.

24. A method of inducing an immune response in a subject, said method including the step of administering to the subject an effective amount of an immunogenic composition comprising:
a plurality of erythrocytes;
a plurality of erythrocytic organisms; and
an antigen masking agent conjugated, linked or otherwise associated with the erythrocytes.

25. (canceled)

26. A method of producing an immunogenic composition, said method including the steps of:
(a) providing a plurality of erythrocytes that contain or are infected with an erythrocytic organism; and
(b) contacting the erythrocytes with an antigen masking agent for a period of time and under conditions to facilitate the antigen masking agent conjugating, linking or otherwise associating with the erythrocytes.

27-30. (canceled)
